(19) United States
(12) Patent Application Publication
(10) Pub. No.: US 2012/0101033 A1
(43) Pub. Date: Apr. 26, 2012

(54) RETINITIS PIGMENTOSA TREATMENT

(76) Inventors: Totada R. SHANTHA, Stone Mountain, GA (US); Jessica SHANTHA, Stone Mountain, GA (US)

(21) Appl. No.: 13/339,327

(22) Filed: Dec. 28, 2011

Related U.S. Application Data
(63) Continuation-in-part of application No. 12/898,524, filed on Oct. 5, 2010.

(51) Int. Cl. A61K 38/30; A61P 27/02

(52) U.S. Cl. 514/6.5

(57) ABSTRACT

A method of treatment of retinitis pigmentosa using a medically effective dose of insulin, IGF-1, and chlorin e6 topically applied to the conjunctival sac of the afflicted eye. The combination of these is very effective in treating retinitis pigmentosa and may be repeated as directed by a medical practitioner. The method includes preparing the dosage and filling an eye dropper with the compound, then having the patient lie in a supine position while administering the dosage. The patient remains in this position for 5 minutes to ensure absorption of the compound. In one embodiment, single use eye droppers are provided to simplify treatment. The particular dosage is adjusted to take individual metabolisms into account. A thorough examination of the patient's eyes should be done prior to treatment.

### CROSS REFERENCE TO RELATED APPLICATIONS

This is a continuation in part of U.S. patent application Ser. No. 12/898,524, filed Oct. 5, 2010, the entire content of which is hereby incorporated by reference.

### BACKGROUND OF THE INVENTION

Retinitis pigmentosa (RP) is a devastating eye condition affecting the retinal rods. This is an inherited disorder, in which the photoreceptors rods gradually degenerate and become dysfunctional. The chief function of the retina is transduction (conversion) of light into nerve impulses by the rods and the cones. Retinitis pigmentosa is a chronic retinal degeneration where the deterioration be associated with by abnormal deposits of pigment in rods of the retina. The disease causes a progressive decrease in peripheral vision, which this type of vision is the side vision. Eventually, the person with retinitis pigmentosa can see only straight ahead which the patient experiences a condition known as “tunnel vision” with night blindness. There is insufficient knowledge about the causes, the progression, and the treatment of RP.

Retinitis pigmentosa (RP) is a group of inherited diseases that damage the light-sensitive rods and the cones that make up the outer layers of the retina. Rods provide side (peripheral) and night vision and pathologically affected more than the cones. The cones are concentrated in macula called fovea centralism provides color and clear sharp central vision. The fovea vision is necessary in humans for reading, watching television, driving, and with other activities where visual detail is required. The fovea centralism includes Para fovea, per fovea of macular regions. Macula luteal is devoid of blood vessels where the macula luteal receives oxygen and nutrition from choroid BV, across the Bruch's membrane, and retinal pigment epithelium (RPE).

The prevalence of retinitis pigmentosa (RP) in The United States is about 1 in 4000. The worldwide prevalence of RP is about 1 in 3000 to 1 in 5000. The carrier state is approximately one in 100. The highest reported incidence of occurrence for RP is among the Navajo Indians where and the lowest is in Switzerland. A multicenter population study of retinitis pigmentosa population is 45 years or older found that 52% had 20/40 or better vision in at least one eye, 25% had 20/200 or worse vision, and 0.5% had no light perception. RP can also afflict from infancy to the mid-30s to 50s. The X-linked RP gens are expressed in male only. These X-linked varieties indicate that men are afflicted more than women are.

LIGHT PERCEPTION BY PHOTORECEPTORS

The retina is the light (photon) sensitive portion of the eye, which the retina contains the photoreceptors (cones and rods) and are the photosensitive cells of the eye for detecting the light that we see. They perform light perception by use of light sensitive pigments. The light sensitive pigments are made of protein called opsin and a chromophore called retinene, which the variant is of vitamin A. The rods contain rhodopsin that the pigment is in the rods. The cones contains iodopsin which the cones have three distinct photo pigments. The rods and cones respond to light where they transmit signals through successive neurons that trigger a neural discharge in the output cells of the retina and the ganglion cells. The visual signals are conveyed by the optic nerve to the lateral geniculate bodies from where the visual signal is passed to the visual cortex (occipital lobe) and registered as a visual stimulus.

It is possible that the photoreceptors are genetically defective where the photoreceptors produce large amounts of reactive oxygen species (ROS) which the ROS could not be mopped out of the retina due to untimely or reduced supply of ATP from the mitochondria. It is likely that the genetic defect in retinitis pigmentosa is in the mitochondrion, in which the mitochondrion does not supply needed ATP to reconstitute the photo pigments and to pump out ROS. The result is ROS accumulation and inducing damage to photoreceptors. In one sense, retinitis pigmentosa can be called as a mitochondrial disease. A significant part of this invention is that the insulin, IGF-1, chlorin e6, antioxidants, and reducing the temperature of the eye ball during sleep will restore the mitochondrial function by metabolic effects of rebuilding all the organelle which the intracellular organelle includes the mitochondrion, the endoplasmic reticulum, the Golgi apparatus, the lysosomes, and the nucleus.

Retinitis pigmentosa (RP) is not a single disease but a collection of genetic eye conditions with symptoms of night blindness which precedes tunnel vision for many years due to progressive retinal dystrophy with rods reduction due to apoptosis which can lead to blindness. Many people with RP will not become legally blind, until, they are in their 50s where these individuals maintain a quantity of sight all of their lives. Others go completely blind from RP where some cases result with blindness in early childhood. Development and progression of RP is different in each case. For the first time, the Retinitis pigmentosa afflicted experience defective darkness adaptation or nyctalopia (night blindness) followed by reduction of the peripheral visual field (contributing to the term known as tunnel vision)

SIGNS OF RETINITIS PIGMENTOSA

Detailed Ophthalmological examination reveals the mottling of the retinal pigment epithelium with black bone like spicule pigmentation is pathognomonic of retinitis pigmentosa. Ocular features include waxy pallor appearance of the optic nerve head, thinning of the retinal blood vessels, cellophane maculopathy, and cystic macular edema where, subsequent, posterior sub capsular cataract may occur. The condition can be associated with other oculopathies.

DIAGNOSIS OF RETINITIS PIGMENTOSA

The diagnosing of retinitis pigmentosa relies on the examination of fundus of the eye (characteristic pigment spots), the visual field (to evaluate the sensitivity of the various parts of the retina to light stimuli), electroretinogram, fluorangiography, and visus examination. The electroretinogram (ERG) consists of recording the electrical activity of the retina in response to particular light stimuli. The ERG makes possible distinct valuations of the functionality of the two different types of photoreceptors Cones and rods. The electroretinogram is necessary for diagnosing retinitis pigmentosa when the illness is in its initial stages. The fluorangiography test; make capillaries and the veins visible and reveal their functional state of their walls. Visus examination permits a valuation of visual acuity consists of the patient reading letters of different sizes at a distance of three meters.

RETINITIS PIGMENTOSA AND THE GENETIC ORIGIN

Studies have shown that the retinitis pigmentosa is caused by mutations in the rhodopsin gene, the peripherin gene, and possibly in other genes within the rods. Mutations in the peripherin gene may be the cause of another devastating retinal disorder namely “macular dystrophy.” RP can be inherited in an autosomal dominant, autosomal recessive or X-linked manner. One of the main biochemical causes of RP in the case of rhodopsin mutations is protein misfolding. In addition, molecular chaperones are also involved in RP. This invention will help to prevent or delay the misfolding of the outer segment involved in RP.

PATHOPHYSIOLOGY OF RETINITIS PIGMENTOSA

Retinitis pigmentosa pathogenesis and its treatment are elusive. All we know is the RP is typically a rod and cone dystrophy of the retina. The genetic defects cause cell death (apoptosis) concentrated in the rod photoreceptors; whereas the cell death is less in cones. There are about 120 million rods and 7 million cones in each eye that the disease can affect. There is shortening of the rod outer segments followed by loss of the rod especially in the mid periphery of the retina. The rods have a propensity to be worse in the inferior segment of the retina. This finding suggests a role of constant light exposure, where the relation to the production of oxidant reactive oxygen species (ROS) which migrate to the inferior segment (Gravitational pooling of the metabolites), resulting in the death of the photoreceptors due to their effect. The rods are densely packed and more in number in the midperipheral retina. The cell loss in this area tends to lead to peripheral and night vision loss. Cone photoreceptor death occurs in a similar manner to rod apoptosis with the shortening of the outer segments resulting in cell loss where only a few cells are affected.

RETINITIS PIGMENTOSA CLINICAL HISTORY

The initial symptom in RP is night blindness (Nyctalopia), which is a painless, progressive and is considered a feature of the disease. Patients might struggle with tasks at night or in dark places. There is a problem walking in dim lit rooms (e.g, movie theaters), difficulties driving in low light, sundown, misty cloudy conditions where the individual needs a prolonged period of time needed to adapt from light to dark. In the early stages, the peripheral vision loss is often asymptomatic. Some patients reports as tunnel vision. Such patients may report running into furniture or door frames. The patients struggle with sports such as tennis, softball, football, basketball where periphereal vision is required and also see flashes of light (photopsia). Rule out phenothiazines/thioridazine toxicity to diagnose retinitis pigmentosa.

PHYSICAL FINDINGS IN RETINITIS PIGMENTOSA UPON EXAMINATION

The common findings on examination are Vision changes such as Snelling visual acuity can vary from 20/20 to light perception, but this is usually preserved until late in the disease. Pupil reaction can be normal or a lacking defect. Surprisingly, nearly 50% of adult patients develop posterior sub capsular cataracts. This is a hint that there is oxidative damage due to generation of free radicals by the light in both these conditions. The retinal fundus show Bone spicules—Midperipheral retinal hyper pigmentation in a characteristic pattern; Optic nerve waxy pallor appearance; Atrophy of the retinal pigment epithelium in the mid periphery of the retina with retinal arteriolar attenuation; loss of the foveolar reflex or an abnormal vitreoretinal interface. Retinitis pigmentosa can be associated with cone-rod retinal degenerations present with central macular pigmentary changes (bull's eye maculopathy).

PRESENT TREATMENT AVAILABLE FOR RETINITIS PIGMENTOSA

There is not a specific cure for the RP condition. The progressive evolution of the retinitis pigmentosa reduced by the daily intake of 1 5000 IU (equivalent to 4.5 mg) of vitamin A palmitate (Berson E L, Rosner B, Sandberg M A, et al. (1993). “A randomized trial of vitamin A and vitamin E supplementation for retinitis pigmentosa”. Arch. Ophthalmol. 111 (6): 761-72). Recent studies have shown that appropriate vitamin A supplementation can postpone blindness by almost 10 years, but it is not a cure (Berson EL (2007). “Long-term visual prognosis in patients with retinitis pigmentosa” the Ludwig von Sallmann lecture Exp. Eye Res. 85 (1): 7-14). Scientists continue to investigate possible treatments without much success. Future treatments may involve retinal transplants, artificial gene therapy, stem cells, other nutritional supplements, and/or drug therapies. This invention brings therapy in which the therapy will hold back the photoreceptors' degeneration and the progression of the disease and curtail or cure the condition.

Scientists at the Osaka Bioscience Institute have identified a protein, named Pikachurin, which they believe could lead to a treatment for retinitis pigmentosa (Sato S, Omori Y, Katoh K, et al. (August 2008). “Pikachurin, a dystroglycan ligand, is essential for photoreceptor ribbon synapse formation”. Nat. Neurosci. 11 (8): 923-931). Attempts have also been made at University College London Institutes of Ophthalmology to treat retinitis pigmentosa with stem cell implant in mice resulting in photoreceptors development with the necessary neural connections. Previously, there was belief that the mature retina has no regenerative ability. These modalities are not available to treat retinitis pigmentosa in humans and may be not be applicable in human retina.

THE FOLLOWING ARE SOME OF THE THERAPIES DESCRIBED IN VARIOUS PATENTS

U.S. Pat. No. 7,037,943 B2, U.S. Pat. No. 5,948,80, U.S. Pat. No. 6,716,835 U. S. Patent Application Publication Number: 2001/0049369 A1, U. S. Patent Application Publication Number: 2009/0060980 A1 Disclose methods of treatment of retinitis pigmentosa. The US patent publication 2010/0330042 A1 (WO 2007/135220 A1) issued to DR. LA ROSA CANO et al, describe the use of proinsulin with activity enhances preparation for treating retinitis pigmentosa in experimental animals. The object of the invention by Cano is aimed at a pharmaceutical composition of the invention in which the compound that induces the activity of proinsulin is a protein or peptide encoded by the proinsulin sequence, genetic construct or vector of the invention. This invention does not use insulin, IGF-1, and chlorin e6; instead describe proinsulin and its enhancers. Proinsulin is 90% less potent compared to the insulin. None of these patents discloses the use of insulin, IGF-1, and chlorin e6 with antioxidants and other Adjuvant treatment modalities to treat retinitis pigmentosa as described in the present invention.

In an ingenious vitro studies, this effect of augmentation and amplification effects was shown, in that the insulin activates and modifies metabolic pathways in MCF-7 human breast cancer cells by paracrine, and intracrines effects. The insulin (so also IGF-1) increases the cytotoxic effect of methotrexate up to 10,000 (ten thousand times-augmentation and amplification effects) folds (Oliver Alabaster' et al. Metabolic Modification by Insulin Enhances Methotrexate Cytotoxicity in MCF-7 Human Breast Cancer Cells, Eurj Cancer Clinic; 1981, Vol 17, ppl 223-1 228). Our studies supports the findings of Alabastor (IBID) that the disease or the healthy cell sensitivity to the therapeutic and biological agents as those to be used to enhance night vision and treat night blindness in the presence of insulin (Shantha T. R., Unknown Health Risks of Inhaled Insulin and IGF-1. Life Extension, September 2007 pages 74-79, Post publication comments in September 2008 issue of Life Extension, Pages 24. Shantha T. R and Jessica G. Inhalation Insulin and IGF-1, Oral and Nasal Insulin and IGF-1 Sprays for Diabetics: Panacea or Evolving Future Health Disaster. Part I: Townsend Letter Journal: Issue #305, December 2008 pages: 94-98; Part II: Townsend Letter, January 2009, Issue # 306, pages -106-110).

Insulin and IGF-1-like have similar three-dimensional structures and similar in function. IGF-1 is a single-chain polypeptide of 70 amino acids. IGF-1 is a trophic factor similar to insulin that circulates at high levels in the blood stream and mediates many effects of growth hormone. Although the main source of IGF-1 in the serum is the liver, many other tissues synthesize it and are sensitive to its trophic action.

IGF-1 maneuvers and influence neuronal structure and functions throughout the life span. In an important experiment, Zheng et al showed the role of IGF-1 has regenerative growth effects in the inner ear epithelial cell culture growth (Zheng, J. L., Helbig, C. & Gao, W-Q. Induction of cell proliferation by fibroblast and insulin like growth factors in pure rat inner ear epithelial cell cultures. J. Neurosci. 17:216-226 (1997). This study showed that the IGF-1 has the ability to preserve nerve cell function and promote nerve growth in experimental studies. The retina is nothing but the extension of the brain; hence, the effect of these therapeutic agents on the retinal is similar to the effects on the CNS. Therefore, the IGF-1 and insulin play an important role in maintaining proper integrity, growth, repair, regeneration, and functioning of the eyes and retinal photoreceptors in particular. Because of these properties, recombinant human IGF-1 is in clinical trials for the treatment of amyotrophic lateral sclerosis (ALS) which acts as trophic factor on motor neurons and prevents their apoptosis. The primary function of IGF-1 is to stimulate cell growth. Cells in every part of the body benefit from IGF-1. Its effect in the treatment of night blindness of retinitis pigmentosa can have therapeutic implication when combined with chlorin e6 and insulin. They can augment and amplify the effects of chlorin e6 and other Adjuvant therapeutic agents in combination as ophthalmic drops or similar to insulin.

There is a clear indication that insulin and IGF-1; not only plays a role in potentiation of (augmentation/amplification effects) the therapeutic, pharmaceutical, biochemical, and biological agents or compounds; they also enhance their uptake. They can also independently stimulate cells growth in eye structures (as it happens in the inner ear epithelial cells which are akin to photoreceptors) including retinal cells particularly photoreceptor.

The insulin and IGF-1, induces cell growth, mitosis, enhances metabolism, increases the glutathione synthesis needed for health (besides glucose transport) of the photoreceptors. This enhanced mitosis, increases the production of nuclear proteins in the nucleus and ribonucleoprotein production by the endoplasmic reticulum, activates the Golgi complex; enhances the lysosomes activity. Thus, the insulin and IGF-1 helps to break up endocytosed toxic substances, cellular debris, and to eliminate the cellular toxins within the photoreceptors cells (augmentation/amplification effects).

The insulin, and IGF-1 deposited in the conjunctival sac, will enhance the uptake of antioxidants and other therapeutic, pharmaceutical, biochemical and biological agents or compounds by the dysfunctional cells of the retina. They mop up the ROS to prevent further damage to the rods (cones) and to restore the function of the retina in retinitis pigmentosa described in this inventive method (Shantha, T. R. Site Of Entry Of Rabies Virus Form The Nose And Oral Cavity; And New Method Of Treatment Using Olfactory Mucosa And By Breaking BBB, presented at The 2^{nd} International Rabies In Asia Conference Held In Hanoi, 2009, Pp 70-73, and The Rabies in the North Americus (XX RITA), held in Quebec City, 2009, Pp 20-21, Rabies Cure: United States Patent Application Publication No.: US 201110020279 A1 rabies cure, Totada R. Shantha). Thus, the present inventive method not only enhances the uptake of therapeutic agents, but also enhances their therapeutic effect inside the photoreceptors afflicted cells as reported by Alabaster (IBID).

U. S. Patent Application Publication Number:2010/0330042 A1 (WO 2007/135220 A1) issued to DR. LA ROSA CANO et al, describe the use of proinsulin with activity enhances preparation for treating retinitis pigmentosa in genetically modified experimental animals. Their invention relates use of compounds that induces the activity of proinuslin. They do not discuss the use of insulin, and IGF-1 in combination to treat human retinitis pigmentosa. It is important to note that the insulin itself is a trophic factor, but instead they use proinuslin with activity enhancers that have 90% less activity compared to insulin. A particular object of their invention based on the use of a compound that induces the activity of pro insulin in which the inducer compound is a nucleotide sequence, which allows the expression of a neuroprotective protein or peptide.

Further, they do not use IGF-1, which is much more trophic to neuronal tissue and has regenerative effect on the neuronal ectodermal cells such as photoreceptors and hair cells of the inner ear that are undergoing apoptosis. Proinsulin synthesized in the endoplasmic reticulum of islets of Langerhans, where it is folded, and its disulfide bonds are oxidized. It is, then transported to the Golgi apparatus, where it is packaged into secretory vesicles, processed by a series of proteases to form mature insulin. Fully formed insulin has 35 fewer amino acids; 4 of the amino acids are removed altogether, and the remaining 31 form the C-peptide. Due to less amino acids content, it is smaller molecule than the proinsulin. Hence, it is more readily absorbed from the conjunctional sac. To form insulin from proinsulin, the C-peptide is abstracted from the center of the proinsulin sequence; the two other ends (the B chain and A chain) remain connected by disulfide bonds. Because of the complex biochemical event to transform proinsulin to insulin, the damaged photoreceptors cells may not have the capacity to convert it fully to functional insulin to repair the photoreceptors. Even if they do, the photoreceptors have to expand more energy with production of ROS in this physiological event. Hence, we used genetically synthesized insulin, which is akin to human pancreatic insulin and does not have to undergo transformation to insulin to be therapeutically more effective compared to proinsulin. It is a known that the proinsulin may cause the body to react with a rash, resist the circulating insulin, or even make dents or lumps in the skin at the site of proinsulin was injection. We want to avoid such reaction by using regular insulin.

On 11 Feb. 2009, Orphan designation (EU/3/0B/612) was granted by the European Commission to ProRetina Therapeutics S.L., Spain Drug Company for recombinant human proinsulin for the treatment of retinitis. They have not started clinical trials in patients with retinitis pigmentosa. They did not give such orphan designation to regular insulin, IGF-1, and chlorin e6 described in this invention.

Studies by Sokoloverova et al (1. M. Sokoloverova. A. A. Bochkareva. E. P. Volodina. G. A. Voinov. S. Oleks, and E, Tsinberg. Experimental nol Biologii Meditsiny, Vol. 53, No.4, pp. 64-66. April. 1 962) showed that dropping one drop of insulin (240 units/ml) in the conjunctival sac reduced blood sugar in alloxan induced diabetic rats. They did not find its effectiveness in human retinitis pigmentosa or its augment and amplify the effects on any other therapeutic agents. Importance of this study is that the insulin in the conjunctional sac is readily transported to the retinal and choroidal blood vessels and then to systemic circulation. This study was done five decades back and supports this invention that the insulin, deposited in the conjunctival sac are rapidly absorbed by conjunctival lining, cornea and bulbar conjunctiva. Then transported to the retina, choroid, ciliary body and processes, iris, anterior and posterior chambers of the eye, retro bulbar space and the entire retina including the photoreceptors to recover from retinitis pigmentosa affliction and any pathological state affecting the vision.

Recently, Silvia Corrochano et. al. studied the therapeutic potential of proinsulin (hPi), an antiapoptotic molecule active during retinal development on transgenic mice expressing genetic defect in the skeletal muscle and predisposing them to retinitis pigmentosa like syndrome (Silvia Corrochano,Rima Barhoum, Patricia Boya, Ana I. Arroba, Natalia Rodrfguez Muela, Violeta Gomez-Vicente, Fatima Bosch, Flora de Pablo, Pedro de la Villa, and Enrique,) de la Rosa. Attenuation of Vision Loss and Delay in Apoptosis of Photoreceptors Induced by Proinsulin in a Mouse Model of Retinitis Pigmentosa. Invest Ophthalmol Vis Sci. 2008; 49: 4188-4194). They reported the antiapoptotic activity of proinsulin during retinal development prompted to investigate a possible effect of proinsulin in the photoreceptor apoptosis associated with retinal degeneration in transgenic mice. Their study showed the attenuation of vision loss and delay in apoptosis of photoreceptors induced by proinsulin in transgenic bred mouse model. They did not show such effect on the naturally occurring retinitis pigmentosa in human that could be genetically different compared to transgenic mice response of the genetically bred mice using proinsulin.

These investigators did not use insulin and IGF-1 as described in this invention which is therapeutically 90% more effective compared to the proinsulin. Proinsulin has only 5 to 10% activity (potency) compared to the Insulin, hence therapeutically less effective. Proinsulin has only week affinity for the classic insulin receptor, and it is a poor metabolic potential. It is also noted that the animals treated with proinsulin remained normal glycemic and weight compared to the systemic insulin administration further supporting its week therapeutic effect (Kiug G I, Kahn C R, non-paralled evolution or metabolic and growth-promoting functions of insulin. Nature 81:292:644-646). Further, the proinsulin has only week affinity for the insulin receptors on the cell membranes, making it less effective in the treatment of retinitis pigmentosa. The insulin and IGF-1 receptors are present on the developing and mature retina indicating that the administration of these therapeutic agents as described in this invention is effective. Added to this benefit is that, systemic IGF-I seems to be able to pass the blood-brain barrier (BBB) and to induce neuroprotection, hence reaches photoreceptors in retina and afford protection (Carro E, TrejojjL. J L, Gomez-Isla T, LeRoith 0, Torres-Aleman 1. Serum insulin-like growth factor I regulates brain amyloid-beta levels. Nat Med.2002; 8:1390-1397).

Further these authors (Silvia Corrochano et. al. IBID) did not use IGF-1 or chlorin e6 combination, which are an important nerve growth factor and preventer of night blindness. This invention describes the use of regular insulin and IGF-1, not proinsulin to treat this retinal disease. There is no provision made by these investigators to enhance the night vision, which is one of the most important symptoms (Nyctalopia) of retinitis pigmentosa, as described in this invention.

The studies by Sokoloverova et al (IBID) five decades back and our recent studies have shown that the conjunctiva unlike normal skin does not act as a barrier like stratum corneum of the skin for entry of insulin and IGF-1 due to the paucity of the presence of reduced glutathione. The conjunctiva does not contain much of any insulin and IGF-1-blocking agent. Besides, the conjunctiva does not have the multilayered stratum coneium as seen on the skin that blocks the entry of insulin and IGF-1 from the skin.

There are various forms of insulin used to treat diabetes. Insulin products classified according to their putative action as rapid, short, intermediate, and long acting insulin. We have used rapid acting, short acting, and long acting protamine zinc insulin in our studies. Protamine Zinc Insulin is long acting insulin contains Zinc. Zinc is an antioxidant; hence, this form of insulin is even more effective in reducing the effect of ROS. Because of its zinc content, it is included in compounding of the ophthalmic drops in this invention.

### SUMMARY OF THE INVENTION

A method of treatment of retinitis pigmentosa using a medically effective dose of insulin, IGF-1, and chlorin e6 topically applied to the conjunctival sac of the afflicted eye. The combination of these is very effective in treating retinitis pigmentosa and may be repeated as directed by a medical practitioner. The method includes preparing the dosage and filling an eye dropper with the compound, then having the patient lie in a supine position while administering the dosage. The patient remains in this position for 5 minutes to ensure absorption of the compound. In one embodiment, single use eye droppers are provided to simplify treatment. The particular dosage is adjusted to take individual metabolisms into account. A thorough examination of the patient's eyes should be done prior to treatment.

The present invention describes the retinitis pigmentosa development, signs, symptoms, pathophysioligy and treatments available and new treatment modaliteis of this invention.

One aspect of the present invention is a method for treating the retinitis pigmentosa in humans or animals by administering to the afflicted eye by insulin, IGF-1 and chlorin e6.

In one embodiment of the invention, one or more therapeutic, pharmaceutical, biochemical and biological agents or compounds are used for treatments of retinitis pigmentosa with conjunctival topical administration of insulin, and IGF-1 followed by chlorin e6.

The invention encompasses synergistic combinations which the opthalmic preparation has the insulin, and IGF-1, where the therapeutic efficacy is greater than an additive.

In certain embodiments, the combination therapies encompassed by the invention provide an improved overall therapy relative to administration of an insulin, IGF-1, and chlorin e6 compound with antioxidants.

The invention is for eye drops composition with insulin, IGF-1, and chlorin e6 activity with other therapeutic agent comprising at least one human growth factor selected from the group consisting of basic fibroblast growth factor (bFGF), glial-derived neurotrophic factor (CNTF), pigment epithelium- derived factor (PEDF), glial-derived neurotrophic factor (GDNF), and brain-derived neurotrophic factor (BDNF).

This invention provides a method of applying specially designed cold packs covering both the eyeballs and surrounding bony sockets to maintain a temperature of between 20-22° degrees centigrade during sleep or nap. Along with the application of our therapeutic agents, this physical method reduces the production of reactive oxygen species (ROS) which contribute to the development and progression of the retinitis pigmentosa damaging the photoreceptors.

The present invention provides for a safe and effective ophthalmic insulin and IGF-1 with chlorin e6 preparations combining various therapeutic, pharmaceutical, biochemical, and biological agents or compounds composition used in the treatment of retinitis pigmentosa.

The invention provides eye drops having a composition with insulin, IGF-1, and chlorin e6 activity and absorption enhancers used to relieve the affliction of retinitis pigmentosa.

This invention provides for a method of using the aforesaid composition to treat retinitis pigmentosa using insulin, and IGF-1 with antioxidants, and knonwn therapeutic, pharmaceutical, biochemical and biological agents or compounds already in use for its treament, such a known therapeutic agents agent is vitamin A, E, C, D₃, GLA, omega 3, zinc, and vitamin B6 (pyridoxal phosphate).

This invention provides for a method of using the aforesaid composition to treat retinitis pigmentosa using insulin, IGF-1 and chlorin e6 with antioxidants, and knonwn therapeutic, pharmaceutical, biochemical and biological agents or compounds already in use for its treament, such a known therapeutic agents agent is a cortocosteroids.

This invention provides for a method of using the aforesaid composition to treat retinitis pigmentosa using insulin, IGF-1 , and chlorin e6 with other knonw therapeutic, pharmaceutical, biochemical and biological agents or compounds such as estrogen, tesosterone, and DHEA.

This invention provides for a method of using the aforesaid composition to treat retinitis pigmentosa using insulin, IGF-1 and chlorin e6 with other therapeutic agents known as Bendazac Acetazolamide, protein named Pikachurin, and stem cells.

This invention provides for a method of using the aforesaid composition to treat retinitis pigmentosa using insulin, IGF-1 and chlorin e6 with other therapeutic agents or compounds containing the enzymes glutathione peroxidase (Enzyme A), prolidase (Enzyme B), glucose-6-phosphate dehydrogenase (Enzyme C) and, optionally, aldose reductase (Enzyme D) in aliquot parts and interactive quantities appropriate for administering as ophthalmic drops.

This invention provides for a method of using the aforesaid composition to treat retinitis pigmentosa using using insulin, IGF-1 and chlorin e6 with other therapeutic agents or compounds such as aceto Brinzolmide, calcium channel blocker compound and/or cyclic GMP-dependent channels, namely diltiazem, brimonidine tartrate, a potent alpha-2 adrenergic receptor agonist, and glycan binding protein.

This invention provides for a method of using the aforesaid composition to treat retinitis pigmentosa using insulin, IGF-1 and chlorin e6 with other monoclonal therapeutic agents such as Infliximab (REMICADE™), Etanercept (EMBRESL™), and Adalimumab (HUMIRA™) are to treat autoimmune bodies. Etanercept is a drug used to treat autoimmune diseases by interfering with the tumor necrosis factor (TNF, a part of the immune system) by acting as a TNF inhibitor and we have selected to use it.

This invention provides for a method of using the aforesaid composition to treat retinitis pigmentosa using insulin, IGF-1 and chlorin e6 with other therapeutic agents such as hyaluronic acid (HA), Mitoxantrone (Novantrone), prednisone and intravenous methylprednisolone; Lotemax, an ophthalmic corticosteroid; levocabastine (brand name Livostin); antihistamines (antolozine) together with a medicine that constricts blood vessels (naphazoline, phenylephrine); sodium cromoglycate (4%); non-steroidal anti-inflammatory (NSAID) eye drops; with insulin, IGF-1, and chlorin e6 be used long term to treat retinitis pigmentosa and the allergic conditions.

This invention provides for a method of using the aforesaid composition to treat retinitis pigmentosa using insulin, IGF-1 , and chlorin e6 with other therapeutic agent. These agents are as follows: thylsulfonylmethane (MSM), anethole dithiolethione (ADT-trade name SIALOR), Ethylenediaminetetraacetic acid (EDTA), vitamin A ,and Deferoxamine (also known as desferrioxamine B, desferoxamine B, DFO-B, DFOA, DFB or desferal), and Alagebrium (known as ALT-711).

HISTOLOGY AND PATHOPHYSIOLOGY OF RETINITIS PIGMENTOSA

Anatomic histology and physiopathology of retinitis pigmentosa reveals us that the free radical damage is the important cause and the offender in the development of retinitis pigmentosa affecting mainly the rods, which are already genetically defective. Understanding anatomic histology and pathophysiology will help to understand this invention and the route the therapeutic agent's insulin, IGF-1 , and chlorin e6 pass through and journey to reach the retinitis pigmentosa afflicted rods and the other neuronal components of the retina.

The pathophysiology of the retinitis pigmentosa is not well understood. It is thought to be the result of a defect in the physiological mechanisms of the protection against the photo-oxidative processes involving free radicals (ROS) and possible glutamate damage produced during photo transduction.

It is important to note that the diseases that affect photoreceptors do not damage retinal ganglion cells (which convey the visual impulses from photoreceptors to occipital cortex through lateral geniculate body) and vice-versa. This is because the two layers of retina receive completely independent blood supply and their metabolism with production of ROS separated from each other. Hence, the pathophysiology is restricted to that particular blood supply region. The ganglion cells are nourished by the retinal blood vessels, which originate from the central retinal artery that penetrates the eye through the optic nerve where the artery branches off into smaller vessels to be incorporated into inner blood retinal barrier. These vessels do not play any role in the support of photoreceptors, which are nourished by the underlying choroid by controlled diffusion from choriocapillares passing through the Bruch's membrane and the retinal pigment epithelium (RPE) (FIGS. 4,5,7) through the outer blood retinal barrier.

The retinal pigment epithelium and the Müller cells (radial glial cells of the retina see FIGS. 5, 6) are essential for photoreceptor functioning and homeostasis. The Müller glial cells ensheath the photoreceptors from the synaptic terminus to the level of the photoreceptor inner segment which the glial cells form a very close physical relationship. The relationship is akin to oligodendroglia and the Schwann cells that enclose the axons in central and peripheral nervous system. It is known that the Müller cells offer metabolic and trophic support to photoreceptors and support their continued survival. These cells support photoreceptors and protect the local microenvironment from surplus extracellular potassium and glutamate that accumulates because of the photo transduction cascade and the neurotransmitter releases at the synaptic stations.

The glutamate released at the photoreceptor synapse internalized by the Müller cells. Then by means of high-affinity carrier systems in which the glutamate is converted to the non-toxic amino acid glutamine by glutamine synthetase. This invention of using insulin, IGF-1, and chlorin e6 for the treatment of retinitis pigmentosa helps this biochemical activity of converting oxidant glutamine to glutamate, then to antioxidant glutathione and protects the photoreceptors cells. This enzyme is widely represented throughout the whole group of the Müller cells. This invention of using insulin, IGF-1, and chlorin e6 in the conjunctival sac ophthalmic instillation can help to restore the glutamine synthetase regenerative ability in these overloaded Müller cells and maintain the integrity of the rods preventing and/or delaying their apoptosis as seen in retinitis pigmentosa due to excessive accumulation of glutamate and ROS. Glutamate causes excitotoxicity in the CNS neurons with similar excitotoxicity in the retinal photoreceptors, because the retina is an extension of the brain; no different.

Retinitis pigmentosa is due to damage by ROS. This concept is validated by delay in progression of the disease by the use of Vtiamin A, E, and C and these vitamins are important known antioxidants. Zinc, unlike other metals acts to stop free radical formation by displacing those metals that do have more than one valence including iron. Every time the light meets the photoreceptors, the mitochondria O2 is continuously being formed. This invention of using insulin and IGF-1, with antioxidant and other oral or parenteral antioxidants therapeutic agents mop up these ROS, prevent the photoreceptors damage, and augment the protection of the photoreceptors. Thus our therapeutic approach prevents further damage where by the progression of retinitis pigmentosa. Adding cold packs at night time on the eyes in addition to using the inventive ophthalmic drops reduces the formation of ROS at rest and promotes healing of photoreceptors.

In retinitis pigmentosa, the defense against ROS is inhibited, lacking or missing due possibly due to genetic defect in photoreceptors as well as in the Müller cells. This invention use of insulin, IGF-1 , and chlorin e6 with antioxidants such as Vitamin A,E, C, GLA, Omega 3, carcumin,and Glutathione and other natural supplements can be of immense therapeutic value in treating this condition. Normally, the body can't handle free radicals if antioxidants are unavailable, or if the free-radical production becomes excessive as seen in retinitis pigmentosa due to constant bombardment of light (phtons) on the photoreceptors. The result is damage to the retina and vision.

Free radicals are produced and present in all living cells as a part of the cell metabolic life processes and have a very short half-life, and difficult to measure. The excessive free radicals in cells can attack the cell membranes (the outer coat of the cell and delicate folded lamellae of rods and cones outer segments), break strands of DNA (the genetic material in the cell nucleus) leading to their apoptosis as it happens to photoreceptors.

Experimental studies show that the remaining rods and cone photoreceptors in many retinitis pigmentosa patients function normally for their numbers. This finding support that these photoreceptors can be rescued and protected to maintain acceptable quality of vision (Eliot L. Berson. Retinitis Pigmentosa. The Friedenwald Lecture Investigative Ophthalmology & Visual Science, April 1993, Vol. 34, No. 5, 1659-1676). This invention of using insulin, IGF-1, and chlorin e6 ophthalmic preparations with antioxidants, nutraceuticals and other therapeutic agents (delivered orally, parenteral or conjunctival sac routes) can rescue these remaining photoreceptors, prevent their progression to apoptosis, maintain the remaining vision perceived by these photoreceptors, and prevent the progression of retinitis pigmentosa, which can lead to total blindness.

The retina is an intricate convoluted structure comprising 10 layers of neuronal cell types (FIGS. 5, 7), their synapses, and their axons, as well as the complex Müller glia, and their positioned on retinal pigment epithelium (RPE). The health and the continued existence of the photoreceptors are greatly dependent on the integrity of other surrounding cell types of the retina, especially RPE cells and the Müller cells. RPE-secrete proteins including pigment epithelium-derived factor (PEDF) to promote photoreceptor differentiation and survival of the photoreceptor. This invention with the use of insulin, IGF-1, and chlorin e6 with antioxidant (taken mostly orally, and can be incorporated to ophthalmic drops) will augment the production of PEDF from the RPE cells to maintain the photoreceptors cells integrity and their physiological state.

This invention focuses on saving photoreceptors not affected by the genetic problems of the cones and rods, that the cells can become lethally damaged by a spillover of free radicals and related harmful chemical reactions occurring in the rods. Rods, amacrine, and horizontal cells of the retina undergo neurite sprouting in human retinas with retinitis pigmentosa. These changes in the retinal neurons may contribute to the electroretinographic abnormalities and the progressive decline in vision noted by patients with retinitis pigmentosa. These changes remedied by the use of insulin, IGF-1, and antioxidant delivered to the conjunctional sac as ophthalmic drops.

Photoreceptors are structurally polarized bipolar neurons with one pole of the neuron is the chemical synapse; at the other end is the outer segment, the most highly specialized region of the photoreceptor cells where the Vision originates. This invention of using insulin, IGF-1 , and chlorin e6 and antioxidant will help to maintain the integrity of the retinal pigment epithelium, Müller cells, and the outer segment of the photoreceptors, the most sensitive parts of photoreceptors by providing needed metabolic, nutritional trophic factor support. Insulin, and IGF-1 also facilitates the removal of the ROS and glutamate from the site and supporting physiological functioning of these three structural units.

### BRIEF DESCRIPTION OF THE DRAWINGS

FIG. 1 is a diagram of the longitudinal section of the eye 100 showing conjunctival sac 202 containing the insulin, IGF-1 and chlorin e6 drops.

FIG. 2 is a drawing of the longitudinal section of the eye 200 showing the structures involved in the production and drainage of aqueous humor which transports insulin, IGF-1 and chlorin e6 and other therapeutic agents.

FIG. 3 is a diagrammatic presentation section of the anterior part of the eye 300 presentation of the rich vascular plexus which transports the insulin, IGF-1 and chlorin e6.

FIG. 4 is a diagramatic presentation 400 showing the vascular arrangement of the choroid surrounding the retina.

FIG. 5 is a diagramatic presentation 500 showing the histology of the retina and its blood supply.

FIG. 6 is a diagrammatic presentation 600 showing the histology of the retina, its relation to the blood supply, and the route of Insulin, IGF-1, and chlorin e6 transfer to photoreceptors.

FIG. 7 is a diagramatic presentation 700 showing the conjunctival fornix and the route of drainage of therapeutic agents to the nose.

### DETAILED DESCRIPTION OF THE INVENTION

The ophthalmic drops or preparations used to treat retinitis pigmentosa should be stable, dissolved, or solubilized which the preparation is safe and effective with ophthalmological standards in place.

The following diagrams describes the structure of the eye, and explains the route of movement, transportation, and diffusion of insulin, IGF-1, and chlorin e6, therapeutic agents instilled in the conjunctival sac topically for the treatment of retinitis pigmentosa. With reference now to the various figures in which identical embodiments numbered alike throughout the description of the present invention, described below.

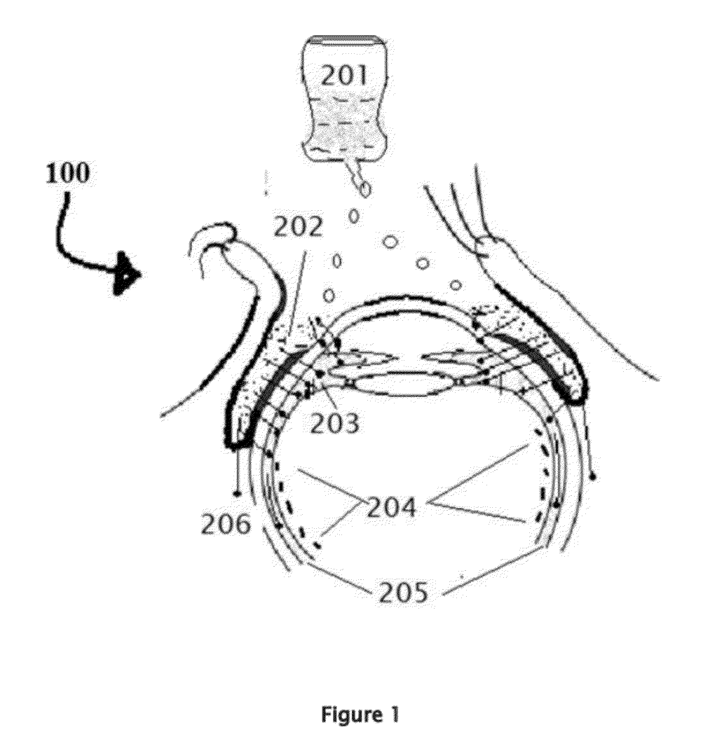

FIG. 1 is the schematic representation of the longitudinal section of the eye 100 showing conjunctival sac 202 where the ophthalmic preparation of this invention, the insulin, IGF-1 and chlorin e6 drops are instilled into the conjunctival sac. The therapeutic agents introduced through a dropper 201 and their passage to iridocorneal angle, anterior and posterior chambers, iris, ciliary body, and processes 203, choroid, and the anterior segment of the retina 204 that contains photoreceptors rods that the photoreceptors affected by the retinitis pigmentosa (drumstick markers). Note that the ophthalmic insulin, IGF-1, and chlorin e6 eye drops, and other therapeutic agents pass on to the choroid 205 adjacent to the retinal pigment epithelium and retinal outer segment of the photoreceptors, delivers the therapeutic agents to the afflicted rods. The therapeutic agents passes through the episcleral plexus of veins to the periphery of the sclera 206, from where the therapeutic agents can be reabsorbed and circulate back into the choroid and retinal blood vessels (BV).

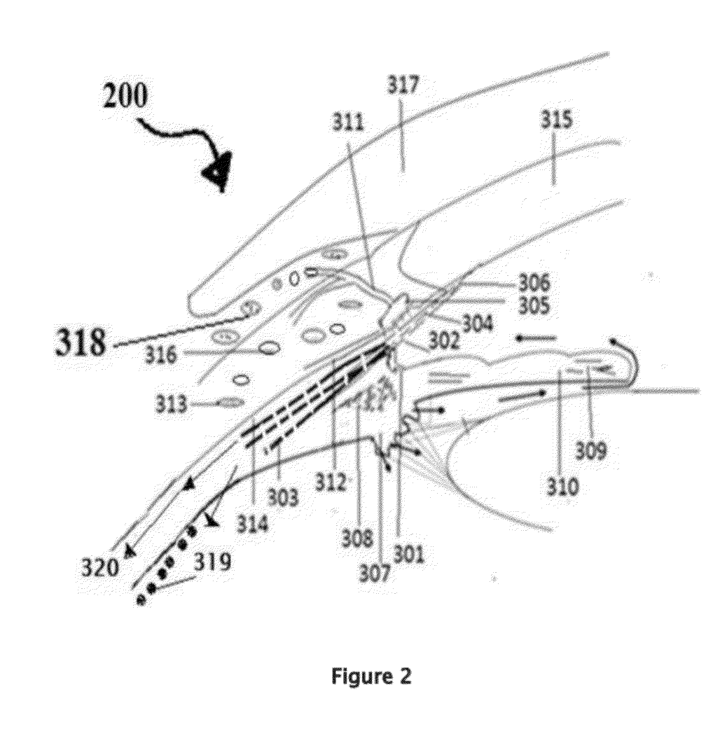

FIG. 2 is the schematic representation of the longitudinal section of the eye 200 showing the structures involved in the production and the drainage of aqueous humor which and the structures pick up and transport the therapeutic agents including insulin, IGF-1, and chlorin e6 used in the treatment of retinitis pigmentosa of this invention. The insulin, IGF-1, and chlorin e6 circulates through various sites of action where the therapeutic agents reach their ultimate site of action with ease to the retinal rods (arrows). The therapeutic agents entering the anterior chamber aqueous humor, through the episcleral arteriovenous plexus 313,316, 318 pass through the uveoscleral meshwork 301, corneoscleral meshwork 302, juxtacanalicular or cribriform trabecular meshwork 304, Schlemm's canal 305, Corneal endothelium joining the trabecular meshwork 306, Longitudinal 303, and circular fibers of the ciliary muscles 308; muscle fibers of the iris 309, 310, Scleral sinus vein 311, Scleral Spur 312, Scleral Veins 313,316, Suprachoroidal space between choroid and sclera 314. The cornea 315 and sclera 316 participate where the least in therapeutic agent's circulation or transport except at the cornea-scleral junction. The conjunctival sac 317 (fornix) where the insulin, IGF-1, and chlorin e6 and other therapeutic agents or compounds are deposited to be transported (arrows) to the retina through the ciliary body 307, trabecular mesh work, choroid, and irido-scleral angle 301, choroid plexus projecting from the ciliary body 307. The choroid plays an important role in transporting the insulin, IGF-1, and chlorin e6 and other adjuvant therapeutic agents (arrows) to the retinitis pigmentosa afflicted retina 319 (From Shantha T R and Bourne GH. Some observations on the corneal endothelium. Acta Ophthalmologica 41: 683-688: 1963).

This diagram illustrates the ease in which the insulin, IGF-1 and other selected therapeutic agents reach the afflicted retinitis pigmentosa 319 site from the conjunctival sac (arrows) of this invention. From the conjunctival sac 317, the therapeutic agents enter into the anterior chamber, corneal endothelium 306, 304, trabecular meshwork 301, 302, and ciliary body 308, passing through the sub and inter conjunctival blood vessel plexus of the eye 313, 316, 318, choroid 320, suprachoroidal space 314 where they reach their destination 319 to have therapeutic effect on the retina involved in retinitis pigmentosa. The arrows markers indicate the site of entry and the circulation of the insulin, IGF-1, and chlorin e6 therapeutic agents from the conjunctival sac where they exert their effect in the treatment of retinitis pigmentosa.

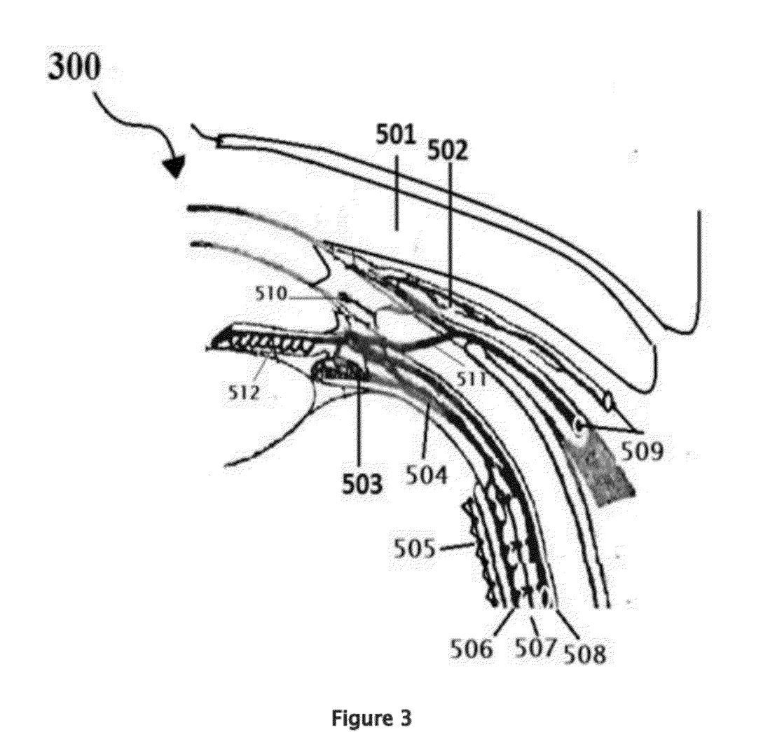

FIG. 3 is a schematic representation of the anteriour part of the eye 300 presentating the rich vascular plexus which the plexus are responsible for transporting the insulin, IGF-1, and chlorin e6 and other therapeutic agents of this invention from the conjunctival sac 501 to the site of retinitis pigmentosa 505. Note the rich vascular plexus 502 under the conjunctiva of the eye ball that transport the therapeutic agents from the conjunctival sac 501 to intrascleral 511 veins and canal of Schlemm 510 with the other venous connection, various vascular structures of iris 512, iridocorneal angle, ciliary body with the ciliary processes 503 where there is a rich BV, and finally passes to the choroid vascular plexus 504, 507, retinal pigment epithelium 506, supra and inter choroidal space 508 where the therapeutic agents reach the base of the rods of the retina 505, the site of the retinitis pigmentosa. Note the rich vascular plexus of the iris 512, choroid, ciliary body 503, that communicates with the subconjunctival BV 502, suprachoroidal space 508, and choroidal vascular network 504,507 which the choroidal vascular net delivers insulin, IGF-1, and chlorin e6 and anti-retinitis pigmentosa therapeutic agents to various structures between the ciliary body and the iridoslceral angle and scleral-corneal space, and supra scleral network of vascular plexus 509 finally reaching the retina. This diagram shows the vascular network under the conjunctival sac delivers the insulin and IGF-1. The therapeutic agents of these inventions are transported to the site of retinitis pigmentosa through various vascular plexus to the afflicted rods, which the defected rods cause the visual disability and blindness in retinitis pigmentosa.

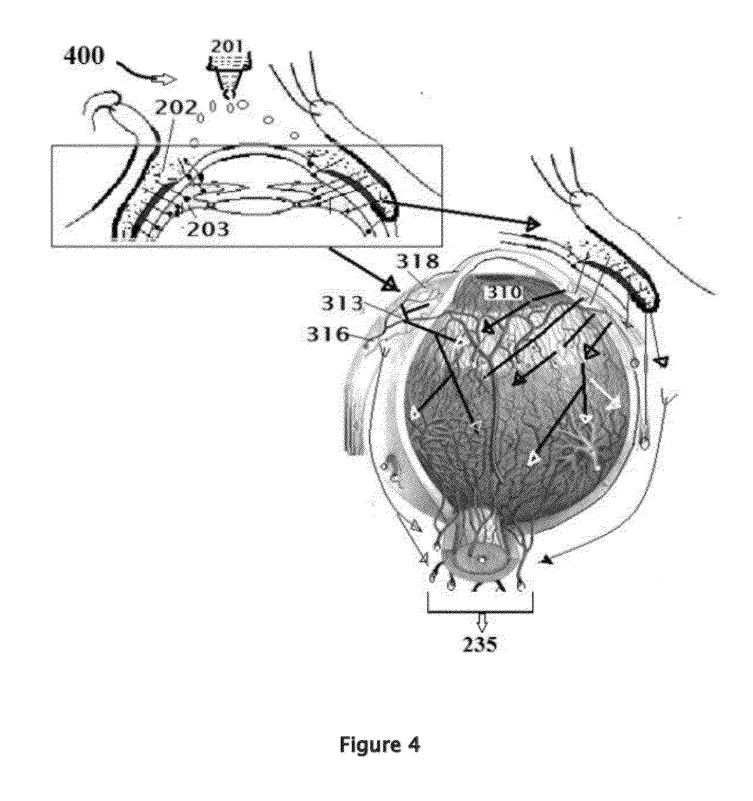

FIG. 4 is the diagramatic presentation 400 showing the vascular arrangement of the uveal track. The uveail track plays an important role in the transport of insulin, IGF-1 and chlorin e6 and therapeutic agents delivered to conjunctional sac. The uveal track is the middle layer of the eye, divided from front to back into, the iris 310, ciliary body 203, and the choroid (arrows) covering the entire retina which are involved in the transport of insulin, IGF-1, and chlorin e6 and other therapeutic agents to the retina where these are the sites of the RP. These three structures of the uveal system are highly vascular and these structures communicate with the subconjunctival 318 and scleral vessels 313,316, 318. The entire uvea is drenched with aqueous humor. The insulin, IGF-1, and chlorin e6 and the adjuvant therapeutic agents 201 from the conjunctival sac 202 are transported to the sub conjunctival venous plexus 318 inter and epi scleral veins 313,316, 318, then the agents are transported to the uveal vascular plexus (multiple drumstick and plain arrows). Through this rich vascular plexus, the therapeutic agents reach the outer segment of photoreceptors of the retina that are located immediately adjacent to the choroid situated on the retinal pigment epitheliums.

The blood vessels of the uvea are involved in the health of the retina by transporting and by providing proper nutraceuticals; at the same time, the metabolites are removed from these photoreceptors. In the same fashion, they carry insulin and IGF-1, and the other therapeutic agents, and deliver to the retinal rods, the site of retinitis pigmentosa. This diagram shows, how efficiently the insulin, IGF-1, and chlorin e6 and the other therapeutic agents from the conjunctival sac 202 are absorbed and transported to the subconjunctival, scleral vascular plexus 318, 313,316; then delivered to the uveal system (arrows) including iris 310, ciliary body and then to the retina, the site of retinitis pigmentosa pathology. Arrows points to the spread of therapeutic agents from the conjunctival sac to the rich uveal vascular network. Long curved arrows shows that some of the therapeutic agents are transported to the supra scleral space where the agents may be transported back through the penetrating arterio-venous network on the optic nerve (arrows) and posterior surface of the sclera (Based on Gray's Anatomy diagram 7.255 on the histology of the eye).

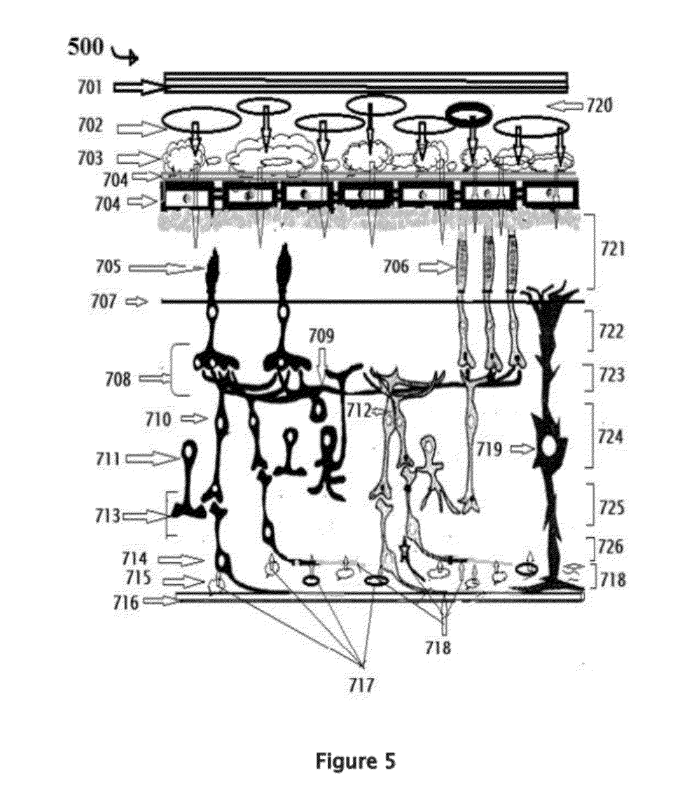

FIG. 5 is the schematic representation 500 showing the histology of the retina in relation to the blood supply. This invention of the use of insulin, IGF-1, and chlorin e6 and other therapeutic agents reach the rod and cone photoreceptors cells involved in the disease of retinitis pigmentosa. It shows sclera 701, large choroidal blood vessels 702, fenestrated choriocapilareis 703 through which the choroidal blood vessels delivers the insulin, IGF-1, and chlorin e6 and the other therapeutic agents (indicated by multiple large and the small arrows directed downwards towards rods and cones) of this invention including oxygen and nutriceticals, through the noncelluar Bruch's membrane 704. The Bruch's membrane acts as a interface between the pigment epithelim 704 and choriocappillaries 703 and separates retinal pigment epithelium form the choriocapilaries 703. The cones 705 are not in intimate contact with the retinal pigment epithelium 704.The rods are in close contact with the retinal pigment epthelium where they brush border 704. The outer limiting membrane 707 formed by the Müller cells 719 separates the photoreceptors outer segments from the rest of the retina in which the separation may prevent the transfer of components from extracelluar space of the photoreceptors to the rest of the retina.

In the same fashion, the therapeutic agents get concentrated as they are transported from choriocapillaries towards the outer segment of the photoreceptors, the site of the retinitis pigmentosa pathology where this invention is very effective. Note the outer plexiform layer 708, and horizontal cells 709 are the laterally interconnecting neurons in the outer plexiform layer of the retina, which the above structures modify and integrate the signals from the rods and cones where the rods and the cones are responsible for allowing eyes to adjust to see equally in bright and dim light conditions. They help to integrate and regulate the input from multiple photoreceptor cells. The bipolar cells 710,712 situated between photoreceptors (rods 706 and cones 705) and ganglion cells 714.

The therapeutic agents from the conjunctiva do not reach these cells in high concentration due to the presence of outer limiting membrane and paucity of vascular network connecting from the choroid system. They act, directly or indirectly, to transmit signals from the photoreceptors to the ganglion cells. Amacrine cells 711 are the interneurons (40 types are recognized) and they are responsible for 70% of input to retinal ganglion cell 714.The bipolar cells 710, 712. are responsible for the other 30% of input to the retinal ganglian cells. The inner plexiform layer 713, ganglion cell layer 714 which the ganglion cell layer receives the signals from the rods and cones. The inner retinal blood vessels 717 where these blood vessels supply oxygen and nutrients to the inner part of retina and the inner retinal blood vessels are shown by mulitiple short arrows pointed towards outer side of the retina. The optic nerve fibers 718 derived from the gangion cells 714 relay the photoreceptors signals to the CNS.

Note the Müller cell 719 contributes to the inner limiting membrane 716 separating the vitreous from the retina and the outer limiting membrane 707. This isolates the sensitive outer segment of the photoreceptors cells of the retina from the rest of the retina. The arrows from choroid indicate the rich vascular supply to the outer segments of the photoreceptors (compared to the rest of the retina), which the outer segments receive the therapeutic agents from the conjunctiva compared to the paucity of BV from the retinal inner BV 717. This diagram shows the insulin, IGF-1, and chlorin e6 and other adjuvant therapeutic, pharmaceutical, biochemical and biological agents or compounds from conjunctiva and chorid blood vessels have easy access to rods 706 and cones 705 outer segments in the treatment of retinitis pigmentosa. The insulin, IGF-1 and other therapeutic agents of this invention are transported by the aqueous humor through the suprachoroidal space which the agents permeate to the space between the retinal pigment epithelium and the photoreceptors.

In one aspect, the trans-conjunctival penetration of insulin and IGF-1, and therapeutic agents facilitated, by adding the absorption enhancers to the therapeutic agents' composition. The enhancers were used to further expedite the entry of these agents to penetrate and to permeate inside the eyeball where the agents are delivered to uveal system, choroid and retina. Penetration enhancers may include anionic surfactants, urea, fatty acids, fatty alcohols, terpens, cationic surfactants, nonionic surfactants, Chitin, DMSO, and other such agents.

The inner limiting membrane 716 is the boundary between the retina and the vitreous body. It is formed by astrocytes, the end feet of Müller cell 719 and it is separated from the vitreous humor by a basal lamina. There may be some leaking of aqueous humor from ciliary epithelium and zonule fibers containing insulin, IGF-1, and chlorin e6 and other therapeutic agents seeping between these two structures through this basal lamina. The transport or soaking has to be minimal. If it does, the concentration is mostly at mid and anterior part of the lower segment (between 5 to 7 O'clock positions) of the retina due to gravitational drag where the pathology of retinitis pigmentosa is prominent at the mid and anterior part of the retina, the main parts affected by the retinitis pigmentosa.

This diagram 500 also shows various histological layers of the retina. They are as follows: layer of retinal pigment epithelium 704, layer of rods and cones 721, outer nuclear layer 722 made up of nuclei from rods and cones, outer limiting membrane 707 formed by Müller cells, outer plexiform layer 723 made up of synapses between the rods, cones with horizontal and bipolar cells. The inner nuclear layer 724 made up of bipolar and amacrine cell nuclei, inner plexiform layer 725 formed by synapses between the ganglion cells 714, 726 and the process of cells from the inner nuclear layer. The nerve fiber layer formed by the axons of the ganglion cells grouped to become the optic nerve where the nerve fiber leaves the eye at the optic disc to lateral geniculate bodies then to the occipital cortex. The inner limiting membrane 716 is made of Müller cells expanded inner feet and astroglia. The diagram shows how each retinal layer is in touch with the blood vessels; their supply of nutraceuticals, oxygen, insulin, IGF-1, and chlorin e6 and other therapeutic agents used in the treatment of retinitis pigmentosa. It is clear that the outer segment of the photoreceptors get the most exposure to the therapeutic agents compared to other functional units of the retina.

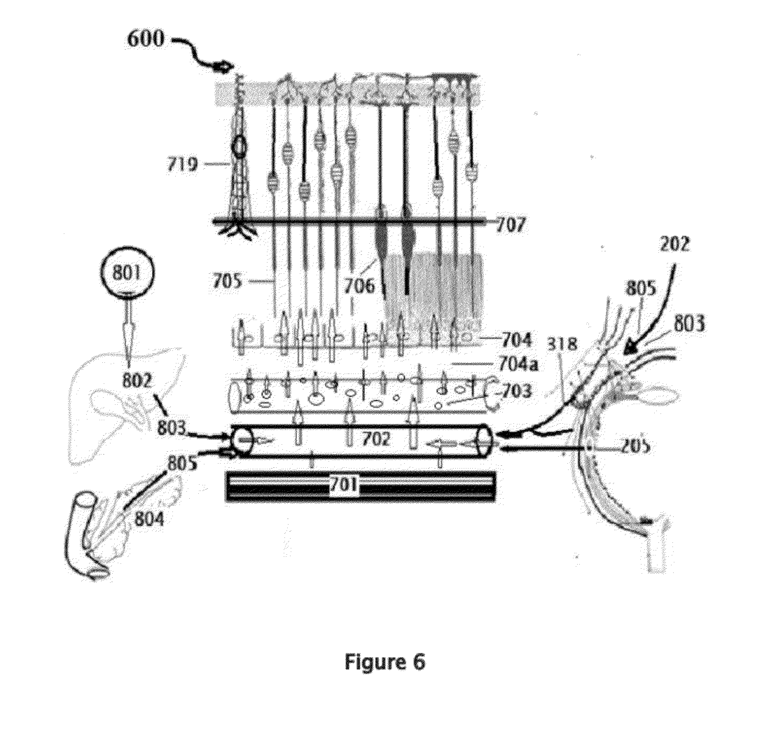

FIG. 6 is the diagrammatic presentation 600 showing the histology of the external layers of retina including photoreceptors. This illustrates the relation to the blood supply to the outer segments of photoreceptors which recives the therapeutic agents delivered through the conjunctional sac. This invention of insulin, IGF-1 and chlorin e6 and other therapeutic agents reach from the systemic blood supply and conjunctival sac of the eyes to reach the rods and cones photoreceptors cells affected in the pathogenesis of the disease retinitis pigmentosa. This diagrm shows sclera 701, large choroidal blood vessels 702, fenestrated choriocapillareis 703 deliver the therapeutic agents insulin, IGF-1, and chlorin e6 805 and other therapeutic agents 803 from the ophthalmic drops 202 instilled into conjunctival sac.

The Liver 802 produces IGFs from growth hormone 801 from the pituitary gland. The pancreas 804, secrets insulin hormone 805, and the insulin hormone enters the circulation through the portal circulation. The ophthalmic drops 202 of this invention in the conjunctional sac 805 and 803, are absorbed by the subconjunctival blood vessels 318 and choroid 205. The growth hormone from the pituitary gland is converted to IGFs, and circulated to reach all over the body including choroidal BV 702, choriocapillares 703 ultimately reaching the retina. The insulin and IGF-1 805 are produced by the pancreas 804 and liver 801; reaches the choroidal BV 205 to reach the retinal photoreceptors 705, 706. The Insulin, IGF-1, and chlorin e6 from the conjunctional sac are transported from the choroidal BV 702 passe to the fenestrated choriocapillares 703 which the choriocapillaries are leaky and the leaked fluid from the inside to extracellular space 707a. This 707a is a cellular Bruch's membrane from this space the Insulin, IGF-1, and chlorin e6 passes through the retinal pigment epithelium (RPE) 704 to reach the outer segments of the photoreceptors 705, 706.

The extracellular fluid is bound by RPE and the external limiting membrane 707 formed by the Müller cells 719. The big and small arrows show the direction of flow of Insulin, IGF-1, and chlorin e6 from the conjunctival sac 202 where there is the systemic circulation from liver and pancreas. The arrows from the choroid indicate the rich vascular supply to the outer segments of the photoreceptors which the photoreceptors receive the therapeutic agents from the conjunctiva. This diagram shows that the therapeutic, pharmaceutical, biochemical and biological agents or compounds from conjunctiva and chorid blood vessels have easy access to rods 706 and cones 705 in the treatment of retinitis pigmentosa. The therapeutic agents are transported by the aqueous humor through the suprachoroidal space where the agents permeate to the space between the retinal pigment epithelium and the photoreceptors.

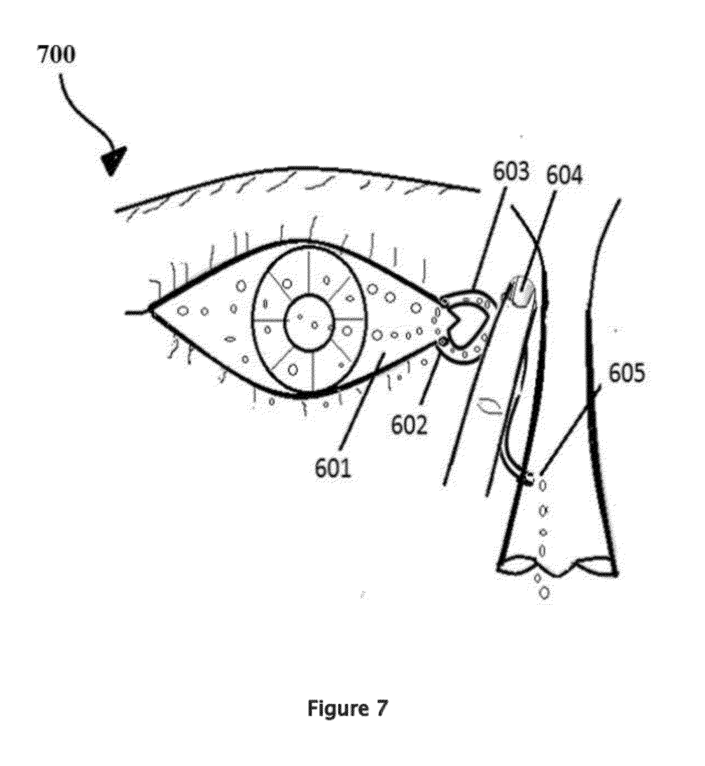

FIG. 7 is the diagramatic presentation 700 showing the route of drainage of the lacrimal fluid and therapeutic agents shown as bubbles from the conjunctival fornix (sac) 601 to the nasal mucosa 605 and illustrates a method to prevent the agents from entering the nasal mucosa. A simple method applying the finger pressure 604 at the medial eye angle and nasal junction. The location of the lacrimal punctum, canaliculi 602,603 and lacrimal sac with a finger 604 will prevent the therapeutic agents drainage to the nasal cavity and the nasal mucosal absorption 605, and their associated systemic adverse effects.

Route of insulin, IGF-1 and chlorin e6 pass through the outer Blood-retinal barrier to reach photoreceptors from the conjunctival sac in the treatment of retinitis pigmentosa

To understand how the therapeutic agents from the conjdunctival sac of the eye reach the retinal photoreceptors, one needs to understand the blood supply of this region, which are responsible for transporting these drugs to the site of pathology. The blood supply to the eye comes from the ophthalmic branch of the internal carotid artery. One of the branches of this artery will become the central retinal artery of the retina and the rest splits to multiple small sized arteries on each side of the optic nerve. These vessels divide into 2 long posterior ciliary arteries and 12-20 short posterior ciliary arteries (FIG. 4-235) that enter the eye immediately adjacent and around the optic nerve. The short posterior ciliary arteries directly supply the choroid and the long posterior ciliary arteries travel in the suprachoroidal space anteriorly then supply the choroid anteriorly via recurrent branches.

Blood in the choroid circulates through the choriocapillaries and larger vessels of the choroid drain into 4-6 vortex veins. The vortex veins (see FIGS. 3,4) emerge just posterior to the equator in eye quadrants (see FIG. 4). The superotemporal and superonasal vortex veins will drain into the superior ophthalmic vein. The inferonasal and inferotemporal vortex veins will drain into the inferior ophthalmic vein (FIG. 4) and will eventually exit via the cavernous sinus. The vortex veins anastomose with the anterior ciliary veins which normally carry blood only from the anterior ciliary muscle.

The circulation of the anterior portion of the eye features an intricately anastomotic system that essentially joins the anterior ciliary circulation and the long posterior ciliary circulation in three interconnected arterial circles ((FIG. 4); 1. an episcleral circle where episcleral vessels join, 2. an intramuscular ciliary circle, and 3. the major arterial iris circle (circumferential vessels in the ciliary body)) with choroidal BV. The anterior ciliary vascular system joins subconjunctival and episcleral vessels at the limbus. These blood vessels are involved in the delivery of therapeutic agents Insulin, IGF-1, and chlorin e6 of this invention to photoreceptors to treat retinitis pigmentosa and other oculopathies from the conjunctival sac.

The Insulin, IGF-1, and chlorin e6 therapeutic agents administered into the conjunctival sac pass through two types of blood-retinal barriers. They are called: 1. Inner blood-retinal barrier (FIG. 5) and, 2. Outer blood-retinal barrier (FIGS. 5, 6).

1. The inner retinal barrier is formed the central artery of retina below the inner limiting membrane embedded in the nerve fiber layer below the vitreous humour in the ganglion cell layer. They play a role in the transfer of therapeutic agents including chlorin e6 administered systemically (IV, subcutaneous) and orally. It is made of a tight junction endothelial cells surrounded by basement membrane, pericytes, astrocytes and Müller cells end feet and is akin to BBB of the CNS blood vessel. It does not play any significant role in transfer of Insulin, IGF-1, and chlorin e6 from conjunctival sac to retinal photoreceptors, the site of pathology in retinitis pigmentosa.

2. The outer blood-retinal barrier is different from the inner blood retinal barrier. They are formed by the multiple posterior, long and anterior ciliary arteries, not one central artery of the retina as seen in inner retinal blood barrier which is akin to BBB. They do have tight junction between the endothelial cells. Then, they give rise to myriads of fenestrated capillaries which leak most of the liquid components of the blood into the space surrounding them into choroid, on the external surface of the Bruch's membrane, and RPE. These liquid components of the fenestrated BV keep the RPE wet and soaked in this media. From here, these leaked nutriceuticals permeate to the outer segments of the photoreceptors to maintain their sturcture and function.

Once these Insulin, IGF-1, and chlorin e6 therapeutic agents and nutritional components leave the fenestrated BV of the inner layers of the choroid, they pass through the Bruch's membrane (acellular) and then tightly connected pigment epithelium to reach the photoreceptors outer segments. It is the one cell layer pigment epithelium with tight junctions which plays a role in outer blood-retinal barrier. The outer retinal-blood barrier delivers the Insulin, IGF-1, and chlorin e6 therapeutic agents and when administered through these two routes. It is the high vasculairty of the choroid (uveal system) which carres therapeutic agents from the conjunctival sac to photoreceptors. The Insulin, IGF-1, and chlorin e6 transported through the transconjunctival, the scleral and choirdal blood vessels; through the multiple posterior, long and anterior ciliary vessels as shown in the FIGS. 3,4 and 5.

Some of the therapeutic agents are also transported through the posterior short ciliary, anterior ciliary and long ciliary arteries when the therapeutic agents are given systemically by parenteral or oral routes.

Both the internal and external retinal blood barriers associated blood vessels are derived from the ophthalmic branch of the internal carotid artery. It is the rich vascular plexus of the uvea (choroid, ciliary body, and iris) and conjunctiva sac (FIGS. 3-5) that plays a role in our method of delivery of Insulin, IGF-1, and chlorin e6 therapeutic agents and their transport. We utilize this rich vascular supply in the present invention to treat night blindness associated with retinitis pigmentosa to enhance the night vision by delivering Insulin and IGF-1 with chlorin e6 therapeutic agents to site of pathology.

The therapeutic agents such as Insulin, IGF-1, and chlorin e6 deposited in the conjunctival sac to treat retinitis pigmentosa do not reach directly into the inner blood-retinal barrier but passes through the outer retinal blood retinal barrier to reach the outer segment of the photoreceptors (FIGS. 1-6). That is why the delivery of therapeutic agents through the conjunctival sac is important and very effective compared to the systemic administration for treating retinal and other ocupopathies including retinitis pigmentosa.

The therapeutic agents from the conjunctival sac (FIGS. 1, 2) have to travel no more than one to two centimeters to reach the site of pathology and need; compared to hundreds of centimeters and meters of blood vessel the insulin, IGF-1 and chlorin e6 as well as other therapeutic agents have to travel to reach the central artery of the retina when administered parenterally. Further, there is no second pass through the liver as happens with oral intake of therapeutic agents, where they undergo biochemical changes and get diluted with 5000 ml of circulating blood. That is why various known Insulin and IGF-1, as well as other pharmaceutical, biochemical, nurticeuticals, and biological agents or compounds have hard time reaching the outer segment of photoreceptors where there are needed to improve the retinitis pigmentosa and alleviate oculopathy when administered IV, IM, subcutaneous and oral routes. The outer segments of the photoreceptors are ideally located right on the retinal pigment epithelium, directly in contact with inner cell membrane of the pigment epithelial cells, hence facilitates rapid transfer of insulin, IGF-1 and chlorin e6 and other adjuvant therapeutic agents in the treatment of retinitis pigmentosa and improving the vision through the conjunctival sac delivery.

MECHANISM OF ACTION OF INSULIN, IGF-1 AND CHLORIN E6 OF THIS INVENTION IN THE TREATMENT OF RETINITIS PIGMENTOSA

INSULIN, IGF-1 AND CHLORIN e6: THEIR BIOLOGICAL EFFECTS ON PHOTORECEPTORS CELLS IN RETINITIS PIGMENTOSA AND THE ROLE THEY PLAY IN THE UPTAKE, DISTRIBUTION; AUGMENTATION—AMPLIFICATION EFFECTS OF THERAPEUTIC AGENTS USED IN THE PRESENT INVENTION TO TREAT RETINITIS PIGMENTOSA.

A variety of carriers, adjuvant agents, absorption enhancers, potentiators of therapeutic action (augmentation/amplification effects), cell metabolic activity enhancers, cell multiplication enhancers, and other methods have been used to enhance the absorption and to potentiate the effect of therapeutic, pharmaceutical, biochemical, and biological agents or compounds administered to the patients for improving the physiological function, and the treatment of diseases. Such endocrine biological agents are Insulin, and IGF-1, used of this invention.

It is known that the Insulin benefits the post ischemic myocardium by stimulating pyruvate dehydrogenase activity. This activity in turn stimulates aerobic metabolism of cardiac and other tissue reperfused. Insulin, and IGF-1, increases the glutathione synthesis by activating gamma-glutamyl-cysteine synthetase, which is a powerful antioxidant. The insulin and IGF-1 metabolic affects reduces both polymorphonuclear neutrophils adhesion due to ROS (reactive oxygen species—ROS—free radicals). Insulin augments the DNA, RNA, and protein synthesis that results in increased growth by mitosis (Osborne C K, et al. Hormone responsive human breast cancer in long-term tissue culture: effect of insulin. Proc Natl Acad Sci USA. 1976; 73: 4536-4540). Insulin and IGF-1 increased redox status by increasing intracellular glutathione (GSH) content in oxidized cells. This reduced the ROS from the cells will cure, and curtail retinitis pigmentosa by mopping the ROS. It enhances the permeability of cell membranes to many therapeutic agents including chlorin e6, neurotrophic agents, and antioxidants. Besides glucose, and electrolytes; Insulin and IGF-1 helps and facilitates to move the drugs and therapeutic agent molecules from extra cellular fluid (ECF) to intracellular fluid (ICE) meaning from outside the cells to inside the cells thus facilitates the uptake of therapeutic agents in the treatment of retinitis pigmentosa.

Insulin and IGF-1 have properties of tissue growth factors, and regulate growth and energy metabolism at the whole organism level farther away from the site of production and application in the conjunctival sac. This is the reason the use of Insulin and IGF-1 with or without adjuvant therapeutic agents topically not only has the local effect; they are absorbed and circulated farther away from the site of application (endocrine effect) and exert their therapeutic effects on the rods, cones, and other neuronal complex in the retina.

Insulin and IGF-1 will exert endocrine, paracrine, intracrines effect (Hernandez-Sanchez C, Lopez-Carranza A, Alarcon C, de la Rosa Ej.de Pablo F. Autocrine/paracrine role of insulin-related growth actors in neurogenesis: local expression and effects on cell proliferation and differentiation in retina. Proc Natl Acad Sci USA. 1995; 92: 9834-9838.), and enhance the absorption, and action of chlorin e6, antioxidants, and monoclonal antibodies and other such therapeutic agents inside the rods to enhance night vision and reduce or eliminate tunnel vision, by maintaining the health of the rods and cones. Once inside the rods and other retinal cells; the insulin and IGF-1 augments and amplifies the effects of chlorin e6 (intracrines effect) and any adjuvant agent proven and/or approved to treat night blindness, and retinitis pigmentosa by restoring their physiological function of the rods. The results show that glutathione (GSH) generation with the help of the insulin can reverse the effect of oxidation (oxidative free radical damage) by tyrosine kinase activation and phosphorylation.

The dose of insulin is typically from 1 to 2 IU per eye per drop. The dose can be decreased or increased depending upon the age, weight, and severity of the retinitis pigmentosa affliction in a given patient.

There is a possibility of developing hypoglycemia when the insulin used as indicated by signs and symptoms such as rapid heartbeat, sweating, dizziness, confusion, unexplained fatigue, shakiness, hunger, feeling hot, difficulty in thinking, confusion. Such patients should be treated with oral ingestion of a fast-acting carbohydrate such as glucose tablets, fruit juice, fruit bowl, chocolate bar, regular Coca-Cola, sugary drinks or eat plain sugar followed with a drink of water or IV administration of 25% glucose, if the reaction is severe.

Insulin, IGF-1, and chlorin e6 are compounded as a liquid ophthalmic isotonic solution containing cyclosporin, or other antiautoimmune therapy agents, or vitamins, and one or more one buffering agents, said buffering agents producing a pH in said composition similar to mammalian eye fluids.

In accordance with one aspect of the invention, the compounds used to apply locally to the eyes site manufactured with physiological saline, oil, gels, slow absorbable gel, patches, solutions, or ointments. The vehicles, which carry these biologically active therapeutic agents, may contain conjunctivally compatible preservatives such as e.g., benzalkonium chloride, surfactants like e.g., polysorbate 80, liposome's or polymers: examples like methyl cellulose, polyvinyl alcohol, polyvinyl pyrrolidone and hyaluronic acid as such. Sterile water or normal saline used in some of the preparations of the eye drops for compounding.

CHLORIN e6 TO IMPROVE DAY AND NIGHT VISION OF RETINITIS PIGMENTOSA PATIENTS:

It was discovered that the porphyrins (chlorphill) are transported to photoreceptors to be utilized effectively enhancing mammalian red light vision (night vision). It is shown that in the presence of various porphyrins in the eyes, and the bleaching of bovine rhodopsin in response to red light is also enhanced leading to conclude that vision enhancement by an unbleachable chlorophyll derivative might therefore be a general phenomenon in vertebrate photoreception (I. Washington, C. Brooks, N. J. Turro and K. Nakanishi, Porphyrins as photosensitizers to enhance night vision, J. Am. Chem. Soc., 2004, 126, 9892-9893. Ilyas Washington, Jilin Zhou, Steffen Jockusch, Nicholas J. Turro, Koji Nakanishi and Janet R. Sparrow. Chlorophyll derivatives as visual pigments for super vision in the red. Photochem. Photobiol. Sci., 2007, 6, 775-779).

Washington et. al. showed that the living rods extracted from a salamander accumulates an exogenous chlorophyll derivative that rendered them as sensitive to red light as they were to green. It is this building block of porphyrins that can be incorporated to retina in the form of Chlorin e6 or other forms of chlorophyll, as they are developed to enhance the night vision and alleviated the night blindness seen in retinitis pigmentosa.

Washington, et al., investigated this premise further, and interestingly their studies showed that the intravenously injected chlorophyll derivative, chlorin e6 accumulates in the eyes of mice and increases the response to red (night vision) or blue light (I. Washington, C. Brooks, N.J. Turro and K. Nakanishi, Porphyrins as photosensitizers to enhance night vision, J. Am. Chem. Soc., 2004, 126, 9892-9893. Ilyas Washington, Jilin Zhou, Steffen Jockusch, Nicholas J. Turro, Koji Nakanishi and Janet R. Sparrow. Chlorophyll derivatives as visual pigments for super vision in the red. Photochem. Photobiol. Sci., 2007, 6, 775-779).

These investigators used water-soluble therapeutically safe chlorophyll derivative “Chlorin e6” for localization in this study. They did not use it by locally instilling in the conjunctional sac with insulin and IGF-1 either. Chlorin e6, a derivative of chlorophyll, is widely used already as a food colorant, a dietary supplement in cancer therapy and for nighttime road illumination. Chlorin e6, a derivative of chlorophyll is prepared from acid or base treatment and/or transmetalation of chlorophyll a. Washington, et al. showed that the chlorin e6 intravenously injected mice eyes had three times red fluorescence (>640 nm) but not in retinas of control mice. These findings showed that the intravenously injected chlorin e6 reaches the retina and remains many hours post administration. Similar observations were also made in the rabbit eye experiments (R. Haimovici, T. A. Ciulla, J. W. Miller, T. Hasan, T. J. Flotte, A. G. Kenney, K. T. Schomacker and E. S. Gragoudas, Localization of rose bengal, aluminum phthalocyanine tetrasulfonate, and chlorin e6 in the rabbit eye, Retina-J. Ret. Vit. Dis., 2002, 22, 65-74).

It is known that both chlorophyll and hemoglobin are structured around a porphyrin ring, the building block of both molecules with the difference being that the core of hemoglobin is iron; whereas, the core of chlorophyll is magnesium. Each has its own unique function, and yet both are very similar in that the chlorophyll is necessary for photosynthesis and hemoglobin in the red blood cell to carry oxygen to every cell in the human body. It is this building block of porphyrins that can be incorporated to retina in the form of Chlorin e6 or other forms of chlorophyll, as they are developed to enhance the night vision and alleviated the night blindness in human as discussed in this invention.

There are many causes of vision deficit and loss in the elderly. The common causes of vision loss are age-related macular degeneration, retinitis pigmentosa, glaucoma, cataract, diabetic retinopathy, and host of other diseases of the cornea, retina, lens, systemic diseases, and the use of certain therapeutic agents (Am Fam Physician 1999; 60:99-108). Reduction of rods and photoreceptors pigment responsible for the night vision is accountable for development of night blindness. The present invention deals with improving the visual pigment in the rods and helps using chlorin e6 instilled into conjunctival sac to improve vision during day and night time (night blindness) in retinitis pigmentosa eyes.

THERAPEUTIC APPLICATION OF INSULIN, IGF-1 AND CHLORIN e6 IN THE TREATMENT OF RETINITIS PIGMENTOSA

ANY TREATMENT OF RETINITIS PIGMENTOSA WITH OPHTHALMIC TOPICAL PREPARATIONS (EYE DROPS) DESIGNED IN THIS INVENTION USING INSULIN, IGF-1 AND CHLORIN e6 ENCOMPASS THE FOLLOWING DESCRIPTION: The ophthalmic preparation is dispensed as eye drops, semi liquids, gels or slow releasing self-absorbing gel fornix insert, ointments as such. They act like a film covering like natural tears over the ocular surface of the exposed eye including conjunctival sac, and cornea.
I. They are formulated to minimize stinging or burning sensations when applied.
II. The ophthalmic drops preparation provides mechanical lubrication for the ocular surface, thus aiding the eyelid to glide easily during the blinking movements.
III. The drops reduce the evaporation of natural lacrimal fluid.
IV. The emulsion, gels, slow releasing self absorbing patch or the watery ophthalmic drops do not react adversely with eye cellular structures, the lacrimal coating, and the eye lid lacrimal glandular system.
V. The compounded Eye drops are stable for a reasonable period at room temperature without refrigeration.
VI. The therapeutic preparations are easily absorbed with or without other absorption enhancers and transported to the site of the pathology,
VII. Besides acting against retinitis pigmentosa pathology, the therapeutic preparations may contain therapeutic, pharmaceutical, biochemical and biological agents or compounds without precipitation or crystallization, at the same time capable of alleviating the underlying cause responsible for RP; at the same time augment/amplify the effects of therapeutic agents with trophic effects when used with this invention to further enhance the treatment efficacy.

The present invention of, insulin, IGF-1, and chlorin e6 therapeutic agents to treat retinitis pigmentosa are compounded to meet all the above recited physiological, pharmacological, therapeutic and safely parameters. Before explanation and description of the disclosed embodiments of the present invention in detail, it is to be implicit that the invention is not limited in its application to the details of the particular examples and arrangements shown; since the invention is capable of other examples and embodiments in treating other oculopathies. The terminology used herein is for the purpose of description and not of limitation.

Insulin and IGF-1-like Growth factor, chlorin e6, and antioxidants have been found to have high therapeutic activity against metabolism of the cells, mopping up of the ROS produced during cell activity and all its function including retina and photoreceptors involved in retinitis pigmentosa. Insulin and IGF-1 restores the proper physiological functioning of the retina by acting against the etiological factors such as ROS, genetic defects, correcting any mitochondrial metabolic defect, and restoring the membrane stability; it enhances the effectiveness (augmentation—amplification effects) of other therapeutic, pharmaceutical, biochemical and biological agents or compounds already used in the treatment of retinitis pigmentosa and other oculopathies. Insulin and IGF-1, of present invention helps to maintain functional and structural integrity of the photoreceptors even though they have genetic defects and helps to delay the expression of genetic defects that these genetic defects exists in the photoreceptors that predispose or cause the retinitis pigmentosa. Further the incorporation of chlorin e6 in this invention, has added benefit of correcting the night blindness (Nyctalopia) and improves the night vision, which is a predominant and may be even the first symptom of retinitis pigmentosa.

EXAMINATION OF THE PATIENTS EYES BEFORE TREATMENT

Before using described inventive methods and examples, a thorough examination of the retinitis pigmentosa affected patient's eye is in order. The examination of the eye may include:
1. Acuity testing
2. Biomicroscopy
3. Intraocular pressure (IOP)
4. Ophthalmoscopy
5. Color vision test
6. Tear osmolality
7. Schimer's test
8. Tear film breakup time (tBUT)
9. Test for Superficial punctate keratitis (SPK)
10. Fluorescein and Rose Bengal staining (RBS) of BV of the retina, as well as cornea, conjunctiva, and eyelids
11. Slit-lamp examination of the conjunctiva, cornea, anterior chamber, iris, and lens
12. The Ocular Surface Disease Index (OSDI)
13. Microscopic examination of the tear filament
14. Maturation index (a Papanicolaous stained sample of conjunctival epithelium)
15. The most important test for retinitis pigmentosa is electroretinogram (ERG) to measure the function of the photoreceptors. In addition, a complete physical examination with blood test for thyroid, parathyroid, growth hormone, insulin and IGF-1, IGF-1, FSH, LH, cortisol, estradiol and testosterone levels, electrolytes, blood cell count, cholesterol levels, ESR, and a urine sample for pregnancy test when deemed necessary when the patient is of childbearing age.

PREPARATION OF PATIENTS FOR THERAPY

To apply this inventive ophthalmic insulin, IGF-1, and chlorin e6 drops, the practitioner and patient will wash their hands with mild antiseptic soap. The person or patient applying the drops must be careful not to touch the dropper tip to the eyelids (and the foreign objects) to avoid contamination if there is eyelids infection. Tilt the head back, or lay down with head extended on a neck pillow in supine position or on reclining sofa; gaze upward and backwards, and pull down the lower eyelid to expose the conjunctival fornix. Place dropper directly over the eye away from the cornea and instill the prescribed number of drops. Look downward and gently close your eye for up to 5 minutes. The patient should try not to blink too much. The patient should not rub the eye. Do not rinse the dropper unless the patient or person knows the sterilization technique in hot water.

If other therapeutic, pharmaceutical, biochemical and biological agents or compounds are to be selected to treat the condition with this invention; wait 5 minutes before using other selected anti-retinitis pigmentosa therapeutic agents or the other variety of ophthalmic medicaments to be added to the present therapeutic modality. It is important to instill medications regularly as prescribed. Consult your doctor and/or pharmacist if the systemic medications that you are taking are safe to use with the eye drops described and prescribed. There is no contraindication for insulin, IGF-1, and chlorin e6 eye drop use except patients with hypoglycemia syndromes, external ocular tumors, sympathetic ophthalmia, and severe eye infections.

To minimize the absorption into the bloodstream and to maximize, the amount of drug absorbed by the eye, the patient should close the eye for five minutes after administering the insulin, IGF-1 , and chlorin e6 drops. Then, press your index finger gently against the inferior nasal corner of your eyelid to close the tear duct, which drains into the nose (FIG. 7). This will prevent any adverse systemic effects due to nasal vascular uptake into the systemic circulation from the nasolacrimal duct drainage of the therapeutic agents from the conjunctival sac.

The anti-retinitis pigmentosa drops get absorbed within 5-10 minutes after application depending upon the therapeutic agents used with the eye drops and concentration of the ingredients. The drops should be applied before bedtime and after rising in the morning. This process can be repeated every 4 to 6 hours, 7 days a week until the desired results are obtained. Retinitis pigmentosa patients can use insulin, IGF-1, and chlorin e6 and other adjuvant antioxidant therapeutic eye drops for long periods. The ophthalmic drops described herein need to be used all through the life in patients with retinitis pigmentosa.

PREPARATION OF INSULIN, IGF-1, AND CHLORIN e6 OPHTHALMIC DROPS TO TREAT RETINITIS PIGMENTOSA

In this ophthalmic preparation of this invention, each ml contains 30 units of insulin, 30 mcg of IGF-1; and 20 mg of Chlorin e6; thus each drop contains 1.5 IU of insulin, 1.5 mcg of Insulin like growth factor-I (IGF-1) and 1 mg of chlorin e6. Prepare 5 ml at each batch of preparation. They can also be prepared in three individual separate bottle dispensers, and used to individually dispense the therapeutic agent one at a time as described below.

A method of treating retinitis pigmentosa consist of the step of topically instilling therapeutically effective doses of insulin, IGF-1 and chlorin e6 to a retinitis pigmentosa afflicted eye's conjunctival sac and their pharmaceutically acceptable salt thereof, wherein the insulin, IGF-1 and Chlorophyll derivative chlorin e6 administered in a dosage prepared as above. The concentration of the therapeutic agents can be increased or decreased depending upon the severity of the condition.

The pH adjusted to prevent the sting when dropped into the conjunctival sac. The preparation can contain nanograms (micrograms) of local anesthetics to prevent the stinging when the eye drops. In pharmacies, a drop use to be another name for a minim, which a drop would be 0.0616 milliliters. The drop standardized in the metric system to equal exactly 0.05 milliliters. The 20 drops make one ml (l cc) which each drop contains 1.5 IU of insulin, 1.5 micrograms IGF-1 and 1 mg of chlorin e6.

Instill two or three drops to each eye lower and upper lid fornix (everted upper eyelid conjunctival sac). The applicant must apply pressure on the nasolacrimal duct as shown in the FIG. 7 to prevent drainage into the nasal cavity.

The data supports the other therapeutic agents which the agents are used after insulin, IGF-1, and chlorin e6, where the agents are prepared in 5-10% solutions of glucose (named as local Insulin and IGF-1 Potentiation Therapy, L-IPT) because the insulin and IGF-1 potentiates the effect of all antioxidants and therapeutic agents used to treat retinitis pigmentosa. Presence of insulin, IGF-1, chlorin e6, and glucose within the photoreceptors can restore function of the various histological components of the retina, choroid, and ciliary body through the replenishment of depleted citric acid. Thus, this invention with the use of insulin, IGF-1, and chlorin e6 with glucose with antioxidant can help in relieving and reversing the retinitis pigmentosa pathology, signs, and symptoms and restore the physiological state to the pigment epithelial cells.

The above pharmaceutical eye drop preparation of this invention may contain antibacterial components; buffering ingredients; a non-noxious pharmaceutical carrier; non-toxic emulsifying, preserving, wetting agents, bodying, viscosity imparting agents, lubricant such as cellulose derivatives (carboxymethyl cellulose), emulsifying agents, hyaluronidase type moister retainer, bodying agents and the like and nonionic surfactant. Furthermore, the appropriate ophthalmic vehicles can be used as carrier media for the current purpose including conventional phosphate buffer vehicle systems, isotonic sodium chloride vehicles, isotonic sodium borate vehicles, and alike.

EXAMPLES OF USING INSULIN, AND IGF-1 TO TREAT RETINITIS PIGMENTOSA

The following are the examples of using this invention of insulin, IGF-1 and chlorin e6 therapeutic agents to treat retinitis pigmentosa

### EXAMPLE 1

Select the patient and establish the diagnosis of retinitis pigmentosa and the RP etiology which the person is suffering. The complete examination of the eye as described above is important, Record the preliminary examination results on the patient chart. The patient will be examined for any corneal and conjunctival and retinal BV afflictions.

Position the patient in supine posture or sitting with head extended with support on a recliner. Using a dropper or dropper squeeze bottle containing the insulin, IGF-1, and chlorin e6 formulations are instilled. Deposit two or three drops of insulin, IGF-1, and chlorin e6 preparation in each eye lower lid fornix and/or everted upper eyelid conjunctional sac. Both eyes receive the eye drops. Apply slight pressure at the nasal angle of eye on the nasolacrimal canaliculi-sac-duct system to prevent leaking of the therapeutic agents to the nose to avoid systemic absorption using the method shown in the FIG. 7.

The patient must remain stationary for at least 5 minutes in supine postion with head extended at the neck. The patient or the caregiver should be trained to apply the ophthalmic drops to the conjunctival sac using sterile methods for the treatment of retinitis pigmentosa with this inventive eye drops containing insulin, IGF-1 and chlorin e6 appropriately. The ophthalmic therapeutic drops are used before going to bed and every 4 to 6 or 8 hoursly aftrer getting out of bed during day time.

While compounding, each of therapeutic agents can be prepared and sealed in a sterile separate dispenser of a 10 ml capacity. Each dispenser contains 150 units of insulin in 5 ml, 150 mcg of IGF-1 per 5 ml; and 100 mg of Chlorin e6 in 5 ml. Thus, each drop containing 1.5 IU of insulin, 1.5 mcg of Insulin like growth factor-I (IGF-1) and 1 mg of chlorin e6 can be dispensed in a separate dispenser. If that is the case, first use the insulin drops, wait for 5 minutes, then administer IGF-1 drops, wait for 5 minutes and finally use chlorin e6 drops. Then wait in supine position with head extended for 5 more minutes to allow the therapeutic agents to be absorbed.

### EXAMPLE 2

Follow the instruction as described in the above EXAMPLE 1. If the men and woman suffer from retinitis pigmentosa with dry eyes syndrome due to estrogen and testosterone deficiency, they need to be treated with estrogen and testosterone ophthalmic along with insulin, IGF-1, and chlorin e6, by oral or parenteral administration. Androgens are trophic factors for various neuronal tissues including retinal photoreceptors. The androgens exert potent anti-inflammatory activity through the production of transforming growth factor beta (TGF-beta), suppressing lymphocytic infiltration, and inflammatory response in the pigment epithelium and the retina and the associated blood vessels. The eye drops containing testosterone can be prepared which the drops used after pretreatment with insulin, IGF-1, and chlorin e6. The ophthalmic drops can be prepared using testosterone (androgen), DHEA—a mild androgen, also.
a. Apply 2-3 drops insulin, IGF-1, and chlorin e6 drop of ophthalmic drops,
b. Wait for 5 minutes,
c. Apply ophthalmic solution of testosterone,
d. Then rest lying down for 5 minutes and then resume,
e. It is important to use these ophthalmic preparations before going to bed, and next day every 4-8 hours.

### EXAMPLE 3

Follow the instruction as described in the above EXAMPLE 1. Previous investigations demonstrated that bendazac prevents protein denaturation produced by U.V. rays. The bendazac is capable of attenuating the biological effects of sun radiations and the tissue associated with adverse effects of ROS on the retina. This possibility was confirmed by the recent observation that bendazac has a protective effect on photo-oxidative processes linked to free radicals involved in the retinitis pigmentosa. The ophthalmic solution of 1% lysine salt of bendazac can be used with muslin and IGF-1. This invention enhances therapeutic agents to reach the site of pathology in the retina. Lysine salt bendazac at the oral dose of 500 mgs/three times daily for a period of 7 or months can also be used in addtion. Insulin, IGF-1, and chlorin e6 combined with bendazac ophthalmic preparations can be compound to be used simultaniously or coupounded separately and then dispensed separately.
a. Apply 2-3 drops insulin, IGF-1, and chlorin e6 drop of ophthalmic drops,
b. Wait for 5 minutes,
c. Apply ophthalmic solution of 1% lysine salt of bendazac,
d. Then rest lying down for 5-10 minutes and then resume,
e. It is important to use these ophthalmic preparations before going to bed, and next day every 4 to 8 hours.

### EXAMPLE 4

The treatment involves the Intravitreal injection of 4 mg (0.1 ml) triamcinolone acetonide to treat macular edema. Intravitreal injections need to be performed by an ophthalmologist under strict sterile environment. This invention avoids this invasive procedure and uses the conjunctival sac corticosteroids locally in the conjunctival sac.
a. Follow the instruction as described in the above EXAMPLE 1
b. Apply 2-3 drops insulin, IGF-1, and chlorin e6 drop of ophthalmic drops,
c. Wait for 5 minutes,
d. The treatment involves the Intravitreal injection of 4 mg (0.1 ml) triamcinolone acetonide to treat macular edema done by an ophthalmologist. Instead of injection, use ophthalmic preparation of triamcinolone drops to avoid invasive procedure. It is compounded by mixing with insulin, and IGF-1 proportion as one ophthalmic eye drop preparation.
e. Then apply insulin, and IGF-1 ophthalmic preparations after 2 hours after injection, then every 12 hours.

### EXAMPLE 5

Follow the instruction as described in the above EXAMPLE 1. We want to compound with Brinzolamide ophthalmic drops incorporated to treat retinitis pigmentosa combined with insulin, IGF-1, and chlorin e6 ophthalmic drops to maintain the integrity of RPE cell layer by decreasing the edema where the relief of the edema can play a role in alleviating the condition of retinitis pigmentosa.

### EXAMPLE 6

Brimonidine allowed for the formation of highly structured photoreceptor outer segments, prevented the expression of stress markers in Müller cells, and preserved the expression patterns of Müller cell markers of proper cell-to-cell contact and differentiation. Ultra structural studies indicated that Brimonidine favored the formation of cell-to-cell junctions between photoreceptor cells and Müller cell with the cell-to-cell junctions indicating that this phenomenon is associated with the action of the neuroprotective effect. Follow the instruction as described in the above EXAMPLE 1.
a. Apply 2-3 drops insulin, IGF-1, and chlorin e6 drop of ophthalmic drops,
b. Wait for 5 minutes,
c. Apply ophthalmic solution of brimonidine tartrate alpha-2 adrenergic receptor agonist as described U. S. Patent Application Publication Number: 2001/0049369 A1,
d. Then rest lying down for 5-10 minutes and then resume,
e. It is important to use these ophthalmic preparations before going to bed, and next day every 4 to 8 hours.

### EXAMPLE 7

Monoclonal antibodies are laboratory created substances that the antibodies can locate and bind to TNF and other cytokines autoantibodies involved in the production of ROS and retinitis pigmentosa. Follow the instruction as described in the above EXAMPLE 1.
a. Apply 2-3 drops insulin, IGF-1, and chlorin e6 drop of ophthalmic drops,
b. Wait for 5 minutes,
c. Apply ophthalmic solution of Monoclonal antibodies. We formulate Etanercept (EMBREL) using 400 μg per ml of ophthalmic solution, which results in 20 μg per drop instilled. The final solution will have insulin, IGF-1, and chlorin e6 as described above to reduce the nonspecific inflammatory processes in the photoreceptors in retinitis pigmentosa caused by ROS,
d. Rest lying down for 5-10 minutes and then resume the activities,
e. It is important to use these ophthalmic preparations before going to bed, and next day every 4 to 8 hourly.

### EXAMPLE 8

Follow the instruction as described in the above EXAMPLE 1. Use the hyaluronic acid (HA) drops along with insulin, IGF-1, and chlorin e6 ophthalmic drops. This compound is one of the most hygroscopic molecules found in nature. Hydrated hyaluronic acid can contain up to 1,000-fold more water than its own weight. These exceptional water retention properties result in enhanced hydration of the corneal surface; retain the active therapeutic agents this invention be slowly released, so that it can be effectively be absorbed, and transported to the site of retinitis pigmentosa.

### EXAMPLE 9

Mitoxantrone is a potent inhibitor of topoisomerase II, an enzyme responsible for uncoiling and for repairing damaged DNA especially in photoreceptors cells of retinitis pigmentosa. Mitoxantrone can be prepared in doses of 100 μg/ ml by premixing with insulin and IGF-1. These drops can be effective in autoimmune related retinitis pigmentosa. Follow the instruction as described in the above EXAMPLE 1.
a. Apply 2-3 drops insulin, IGF-1, and chlorin e6 drop of ophthalmic drops,
b. Wait for 5 minutes,
c. Apply ophthalmic solution of Mitoxantrone can be prepared in doses of 100 μg/ ml. It can be compounded with insulin, IGF-1, and chlorin e6 as one ophthalmic drop,
d. Then rest lying down for 5-10 minutes and then resume.
e. It is important to use this ophthalmic preparation before going to bed, and next day every 4 to 8 hours.

### EXAMPLE 10

Corticosteroid, loteprednol etabonate (LOTEMAX®) Ophthalmic Suspension 0.5% is already available; Pre-treating with insulin, IGF-1, and chlorin e6 can enhance the uptake of these corticosteroids and relive retinitis pigmentosa of the eye. The steroid, insulin, and IGF-1 attenuates the effects of ROS mediated photoreceptor cells, stabilizes the membranes of the photoreceptors and their organelle, which restores function and health. Follow the instruction as described in the above EXAMPLE 1.
a. Apply 2-3 drops insulin, IGF-1, and chlorin e6 drop of ophthalmic drops,
b. Wait for 5 minutes,
c. Apply ophthalmic solution of prednisone, dissolved 4 mg in 1 ml of diluents and then use it with dropper. It can also be formulated with insulin, IGF-1, and chlorin e6 as single dose dispensing ophthalmic drops.
d. Then rest lying down for 5-10 minutes and then resume,
e. It is important to use this ophthalmic preparation before going to bed, and next day every 4 to 8 hours.

### EXAMPLE 11

A study involves the use of desmethyldeprenyl, a metabolite of the anti-Parkinson's drug, deprenyl for retinitis pigmentosa (W. A. Baumgartner, Etiology, pathogenesis, and experimental treatment of retinitis pigmentosa. Medical hypothesis. Volume 54, Issue 5, Pages 814-824. May 2000). Take this oral medication, wait for one hour, and then administer insulin, and IGF-1 ophthalmic drops. Insulin, IGF-1, and chlorin e6 drops will enhance their uptake by the disease-afflicted photoreceptors cells in the eyes as the deprenyl circulates through the choroid and transported through the RPE to the outer segment of the rods photoreceptors.

### EXAMPLE 12

Follow the instruction as described in the above EXAMPLE 1. It is known that many of the neurotrophic factors protect the outer segment of the photoreceptors and prevent their apoptosis. The following are the neurotrophic factors that could be compounded and used to enhance the therapeutic agent's effectiveness with this invention. Such examples are as follows:
a. Apply 2-3 drops insulin, IGF-1, and chlorin e6 drop of ophthalmic drops composition comprising at least one human growth factor selected from the group consisting of basic fibroblast growth factor (bFGF), glial-derived neurotrophic factor (CNTF), pigment epithelium-derived factor (PEDF), glial-derived neurotrophic factor (GDNF), and brain-derived neurotrophic factor (BDNF).
b. Then apply insulin, IGF-1, and chlorin e6 ophthalmic preparations one-hour hours after application of the neurotrophic factors, to enhance their uptake, distribution, and effect on photoreceptors cells.

Follow the instruction as described in the above EXAMPLE 1. Administer insulin, IGF-1, and chlorin e6 ophthalmic drops one to two hours after the oral intake of the following therapeutic agents, vitamins and nutritional supplements. The reports indicate that the progression of the disease can be reduced by the daily intake of 15000 IU (equivalent to 4.5 mg) of vitamin A palmitate. Eleven-CIS Vitamin A can be used for treating this condition (Berson E L, Rosner B, Sandberg M A, et al. (1993). “A randomized trial of vitamin A and vitamin E supplementation for retinitis pigmentosa”. Arch. Ophthalmol. 111 (6): 761-72). Recent studies have shown that the vitamin A supplementation can postpone blindness by almost 10 years (“Long-term visual prognosis in patients with retinitis pigmentosa: the Ludwig von Sallmann lecture”. Exp. Eye Res. 85 (1): 7-14). Using vitamin A with this invention will further add to the therapeutic value of this vitamin uptake and may delay the full blown development of the disease for two decades or more.

Antioxidant superoxide dismutases, catalases, lactoperoxidases, glutathione peroxidases and peroxiredoxins, uric acid, polyphenol antioxidants, and glutathione ophthalmic drops and/or oral administration can also be tired with this invention.

Chelation therapy locally or systemically with Ethylenediaminetetraacetic acid (EDTA), ethyl-sulfonyl-methane (MSM), Alagebrium, and Deferoxamine (also known as desferrioxamine B, desferoxamine B, DFO-B, DFOA, DFB or desferal) will clear these clogged cell layers and photoreceptors cells undergoing changes. They remove any excess iron, calcium, and other metals as well as fatty protenacious deposits that may interfere with photoreceptors physiological role.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection with what is presently deemed to be the most practical and preferred embodiments of the invention. The invention will be apparent to those of ordinary skill in the art that numerous modifications, including, but not limited to, variations in size, materials, shape, form function and manner of procedure, assembly and the use may be made. The preferred embodiment of the present invention is illustrated. The invention should be understood that various changes, and modifications may be made thereto. It should be understood, therefore, that the invention is not limited to details of the illustrated invention. This method can be used to diagnose corneal ulcers; any pathological changes in the cornea and conjunctiva of the eye. The preferred embodiment of the present invention has been described. The invention should be understood that various changes, adaptations and modifications may be made thereto. This should be understood, therefore, that the invention is not limited to details of the illustrated invention examples.

What is claimed is:

1. A method of treating retinitis pigmentosa in a vertebrate comprising the steps of:
preparing a therapeutically effective dose of insulin, IGF-1 and chlorin e6;
filling an eye dropper with said therapeutically effective dose of insulin; IGF-1 and chlorin e6;
placing a patient's head in a supine position;
instilling said therapeutically effective dose of insulin, IGF-1 and chlorin e6 topically to a retinitis pigmentosa afflicted patient eye's conjunctival sac using said eye dropper;
waiting five minutes with said patient's head remaining in said supine position wherein said therapeutically effective dose is absorbed therein; and
repeating said steps in a selected schedule.

2. The method of treating retinitis pigmentosa in a vertebrate according to claim 1, wherein the said vertebrate is a Human.

3. The method of treating retinitis pigmentosa in a vertebrate according to claim 1 is a mammal.

4. The method of treating retinitis pigmentosa in a vertebrate according to claim 1 wherein each milliliter of said therapeutically dose of insulin, IGF-1 and chlorin e6 comprises 20 units of insulin, 20 mcg of IGF-1 and 15 mg of chlorin e6.

5. The method of treating retinitis pigmentosa in a vertebrate according to claim 1 wherein each milliliter of said therapeutically dose of insulin, IGF-1 and chlorin e6 comprises 15 units of insulin, 15 mcg of IGF-1 and 15 mg of chlorin e6.

6. The method of treating retinitis pigmentosa in a vertebrate according to claim 1 wherein each milliliter of said therapeutically dose of insulin, IGF-1 and chlorin e6 comprises 10 units of insulin, 10 mcg of IGF-1 and 10 mg of chlorin e6.

7. The method of treating retinitis pigmentosa in a vertebrate according to claim 1 wherein each milliliter of said therapeutically dose of insulin, IGF-1 and chlorin e6 comprises 30 units of insulin, 30 mcg of IGF-1 and 20 mg of chlorin e6.

8. The method of treating retinitis pigmentosa in a vertebrate according to claim 1 wherein each milliliter of said therapeutically dose of insulin, IGF-1 and chlorin e6 comprises 10 units of insulin, 10 mcg of IGF-1 and 10 mg of chlorin e6.

9. The method of treating retinitis pigmentosa in a vertebrate according to claim 1 comprising a further step of adding a permeation enhancer to said therapeutically effective dose of insulin, IGF-1 and chlorin e6 selected from the group consisting of citric acid, sodium citrate, propylene glycol, glycerin, L-ascorbic acid, sodium metabisulfite, edentate disodium, benzalkonium chloride, chitin and sodium hydroxide.

10. The method of treating retinitis pigmentosa in a vertebrate according to claim 1 comprising a further step of adding a wetting agent to said therapeutically effective dose of insulin, IGF-1 and chlorin e6 selected from the group consisting of polyvinyl alcohol, hydroxyalkyl cellulose, hyaluronidase, methylcellulose and polyvinyl pyrrolidone.

11. The method of treating retinitis pigmentosa in a vertebrate according to claim 1 comprising a further step of cleaning an eye area prior to said step of instilling said therapeutically effective dose of insulin, IGF-1 and chlorin e6.

12. The method of treating retinitis pigmentosa in a vertebrate according to claim 1 wherein said selected interval is 4 times per day.

13. The method of treating retinitis pigmentosa in a vertebrate according to claim 1 wherein said selected interval includes a treatment at bedtime.

14. The method of treating retinitis pigmentosa in a vertebrate according to claim 1 comprising a further step of running an examination of said patient's eyes.

15. A method of treating retinitis pigmentosa in a vertebrate comprising the steps of:
preparing a therapeutically effective dose of insulin and filling a first eye dropper therein;
preparing a therapeutically effective dose of IGF-1 and filling a second eye dropper therein;
preparing a therapeutically effective dose of chlorin e6 and filling a third eye dropper therein;
placing a patient's head in a supine position;
instilling said therapeutically effective dose of insulin topically to a retinitis pigmentosa afflicted patient eye's conjunctival sac using said first eye dropper;
waiting five minutes with said patient's head remaining in said supine position wherein said therapeutically effective dose of insulin is absorbed therein;
instilling said therapeutically effective dose of IGF-1 topically to a retinitis pigmentosa afflicted patient eye's conjunctival sac using said second eye dropper;
waiting five minutes with said patient's head remaining in said supine position wherein said therapeutically effective dose of IGF-1 is absorbed therein;
instilling said therapeutically effective dose of chlorin e6 topically to a retinitis pigmentosa afflicted patient eye's conjunctival sac using said third eye dropper;
waiting five minutes with said patient's head remaining in said supine position wherein said therapeutically effective dose of chlorin e6 is absorbed therein; and
repeating said steps in a selected schedule.

16. A method of treating retinitis pigmentosa in a vertebrate comprising the steps of:
obtaining a single use eye dropper containing a therapeutically effective dose of insulin, IGF-1 and chlorin e6;
placing a patient's head in a supine position;
instilling said therapeutically effective dose of insulin, IGF-1 and chlorin e6 topically to a retinitis pigmentosa afflicted patient eye's conjunctival sac using said single use eye dropper;
waiting five minutes with said patient's head remaining in said supine position wherein said therapeutically effective dose is absorbed therein; and
repeating said steps in a selected interval.
